SEQUENCE LISTING (10 sequences)

SEQ ID NO: 1 - DNA, 1812 bp, Homo sapiens
gttcccaatcagtttccagccaacaccagggtgtcctagtccgcagaggtgtgggggacacactccataatctctacttttctttttgtgcagctgagtcatggagctttcagccccagcacatggctcctccttaactgcgtctgctcaacctccctcagccctgtgaacagcatccccgcacacagacgcagagcaggactctctctgctgccacttcaccttcctgagagaggaccagcggccagagcctcagtgactgccaccctggaggacagggcacaacaaccgtttctggagagaatgggaggattccagaggggcaaatatggaactatggctgaaggtagatcagaagataacttgtctgcaacaccaccggcattgaggattatcctagtgggcaaaacaggctgcgggaaaagtgccacagggaacagcatccttggccagcccgtgtttgagtccaagctgagggcccagtcagtgaccaggacgtgccaggtgaaaacaggaacatggaacgggaggaaagtcctggtggttgacacgccctccatctttgagtcacaggccgatacccaagagctgtacaagaacatcggggactgctacctgctctctgccccggggccccacgtcctgcttctggtgatccagctggggcgtttcactgctcaggacacagtggccatcaggaaggtgaaagaggtctttgggacaggggccatgagacatgtggtcatcctcttcacccacaaagaggacttagggggccaggccctggatgactatgtagcaaacacggacaactgcagcctgaaagacctggtgcgggagtgtgagagaaggtactgtgccttcaacaactggggctctgtggaggagcagaggcagcagcaggcagagctcctggctgtgattgagaggctggggagggagcgagagggctccttccacagcaatgacctcttcttggatgcccagctgctccaaagaactggagctggggcctgccaggaagactacaggcagtaccaggccaaagtggaatggcaggtggagaagcacaagcaagagctgagggagaacgagagtaactgggcatacaaggcgctcctcagagtcaaacacttgatgcttttgcattatgagatttttgtttttctattgttgtgcagcatactttttttcattatttttctgttcatctttcattacatttaaatctctggaccctggagcacttctaatgtatcaccccatggagtcattgttctaataatcaccaattcagactcagatcctcgtggtctatggagcatgctgcttgctgtctgtgcagctcccatttccccttcttcctgatagacttggagctgtgtgcctccactccaaggctgcctgcctgctgtaaacactattccactctgtctgccaacaactgcttcaggaatgggcctgagatcccatgcaggtccctgagaagtgagtaaaagtccgcagaggtggggatggaagatctctccttagatagaacctgtcttcctccctggcattgtggggtctgggcgtgacactgggactctcagcagctttgtgctgccaacctgagattgaaggcagtgcctcagagcagcacagagagttggggccccctgagccctgagccaccagccctgcagcctgccctatctccgcatttccagttgtattagccaatagatttcctacttatttaagctatttgagctccgggtctcttctacctgcattctaaaacattcaaagtaataaaaatttctccacatt

SEQ ID NO: 2 - protein, 307 aa, Homo sapiens
MGGFQRGKYGTMAEGRSEDNLSATPPALRIILVGKTGCGKSATGNSILGQPVFESKLRAQSVTRTCQVKTGTWNGRKVLVVDTPSIFESQADTQELYKNIGDCYLLSAPGPHVLLLVIQLGRFTAQDTVAIRKVKEVFGTGAMRHVVILFTHKEDLGGQALDDYVANTDNCSLKDLVRECERRYCAFNNWGSVEEQRQQQAELLAVIERLGREREGSFHSNDLFLDAQLLQRTGAGACQEDYRQYQAKVEWQVEKHKQELRENESNWAYKALLRVKHLMLLHYEIFVFLLLCSILFFIIFLFIFHYI

SEQ ID NO: 3 - DNA, 4782 bp, Homo sapiens
aagtttttaatgatacctgccgctcaggtggcctaggtggtagtcatgccttgcttctacttacgtagttgtggttctctcttgcctgaactaaagcttgaagagagaacagaatttgctcataggatatgggacacacttcagaaattaggtgctgtgtatgatgtgagtcactataatgctttacttaaagtctatcttcaaaatgaatataaattctcaccaactgatttcctggcaaaaatggaggaagcaaacattcaaccaaatcgagtgacataccagagattgattgcttcttattgtaatgtaggagatattgaaggtgccagcaagattcttggatttatgaaaactaaggatctcccagttacagaggcagtattcagtgcccttgtgacagggcatgccagagctggtgatatggagaatgcagaaaacattctcacagtgatgagagatgccggaattgagcctggtccagacacatacctcgcattattgaatgcatatgctgagaagggcgacattgaccatgttaagcagactctggagaaggtggagaagttcgagcttcaccttatggaccgtgatttactgcaaattatttttagcttcagtaaagctgggtatctcagtatgtctcagaaattttggaaaaagtttacatgtgaaagaagatatattccagatgcaatgaacctcattttacttttagtcactgaaaaattggaagatgtagcgttgcaaattttactagcatgccccgtatcaaaggaagatggcccaagtgtctttggcagtttctttttacaacactgtgtgactatgaatacgcctgtggagaagctaacagactactgtaagaagttaaaggaagtccagatgcactcctttcctctgcagttcaccctccattgtgctttactcgccaataaaactgatttggcaaaagccttaatgaaggctgtgaaggaggaaggttttcctatcagacctcactatttctggccattgctagttggacgtcggaaggaaaaaaatgttcaaggtataattgaaatcctcaaaggaatgcaagaattgggagtacatcctgatcaggaaacatatacagattatgtgattccatgctttgatagtgtaaactcagcacgagccattttgcaggaaaatggatgtctgtctgatagtgatatgttttctcaagctggattgagaagtgaagcagcaaatgggaacttagactttgtattatcatttttgaaatcaaatacattgcccatctcgctgcagtctataagaagtagcctactgctaggcttcaggaggtctatgaatataaatgtttggagcgagataacagaattattgtacaaggatggacgttattgccaggagcctcgaggaccgacggaagctgttggcaattttctttataacttgattgacagcatgagtgactcagaggtacaggccaaggaggagcatttgagacaatacttccatcagctggagaagatgaatgtaaaaattcctgaaaatatctacagaggcattcgtaatctcctggaaagctaccatgttcctgaattgattaaggatgctcacttgttggttgagcgtaagaatttagactttcaaaaaactgtgcaacttacatcatctgaattggagtcaacacttgaaacactaaaagctgaaaatcaacctataagagatgtcctaaagcaactcatattagtgctttgttcagaagagaatatgcaaaaagcccttgaattgaaagcaaaatatgaatccgacatggttactggtggctatgcagctttaataaatttatgctgtcgacatgataaagtagaagatgccttgaacttgaaagaagaatttgaccgcttagattcatctgctgtccttgacaccggcaactatctaggccttgtaagagtattggcaaagcatggcaagctccaagatgctattaagattctgaaggagatgaaagagaaggatgttcttatcaaagatacaacagccttgtcctttttccacatgctaaatggcgcagctttaagaggtgaaattgaaacagtaaaacagttgcatgaagccatcgtgactctagggttagcagaaccatccaccaacataagtttcccattggtcactgtacacttggaaaagggcgacctatctactgctcttgaggtcgccattgactgctatgaaaagtataaagtattaccaaggattcatgatgtcttgtgtaaactggtagagaaaggcgagactgatctaattcagaaagcaatggactttgtgagccaagaacaaggtgaaatggtgatgctctatgatctcttctttgccttcctacaaacaggaaattacaaagaggccaagaagatcattgagactccagggattagagctcgatctgcaaggcttcagtggttttgtgacagatgtgttgcaaataatcaggttgaaactctggaaaaattagtggagctgacacagaagctatttgaatgtgatagagaccagatgtactacaatctgctaaaactgtataaaataaacggtgactggcaaagagctgatgcagtctggaataaaatccaagaagaaaatgttattcctcgtgaaaagacattaagattattagcagaaatccttagagagggtaaccaggaagttccgtttgacgtacctgagttgtggtatgaagatgaaaaacattccctgaattcttcgtcagcctcaaccacagaacctgatttccagaaagatatattgattgcctgccgattgaaccaaaaaaaaggggcatatgatattttcctgaatgcaaaagagcaaaacattgtgtttaatgctgaaacctacagcaatctcattaaattactgatgtcagaagattattttacacaagcaatggaagtgaaagcattcgcggagacccacatcaagggcttcacactgaacgatgctgccaacagccgcctcatcataacgcaagttaggcgggattatttgaaagaggctgtgacaacactgaaaacagtattggatcagcagcagaccccttctaggttagcagtgacccgtgtcatccaggcattggccatgaagggtgatgttgaaaacatagaagtagttcagaagatgttaaatggactcgaagactccattggactttcaaaaatggttttcatcaataacattgctttggctcaaataaagaataataacatagatgccgcaatagaaaacattgaaaatatgcttacttcagagaataaagtcattgaaccccaatacttcggcttggcatacttattcagaaaagtaatagaggagcagttggaaccagcagttgaaaagataagcatcatggcggagagattggccaatcagtttgcaatttataaacctgtcactgattttttccttcaacttgtggatgcaggcaaggtggatgatgccagagctctcctacagagatgtggtgcaattgctgaacaaaccccgattttgttgttgttcctccttaggaattctaggaaacaaggaaaggcatcaactgtgaaatctgtgttagaattgattcctgaattaaatgaaaaggaagaagcatacaattccctcatgaaaagctatgtctcagagaaagatgtcacatctgctaaagcactgtatgaacatttgactgcaaagaatacaaaattggatgatctgtttctaaagcgttacgcatctttgctgaagtatgctggagagcctgtccctttcattgaaccccctgaaagctttgaattttatgcacagcagctaagaaaattgagggaaaactcttcttgaaataaccaggcgatactttgttttgtatatatttgtgattctgtgtctacatgttattttgaagtatatctgagggaaaaataaatgaaaattttctttatgtacttatgtatgtgtgatgcatgttcaaagtcttattgaccataactctgtgcacttggttattggacatttttggagttttttctctgggaaaaatcgatagtgttttcttcaatgctgctgctgtgtgaagccatacttttcaggattcttccctaattggctctttggtttccctgctctgtttcatttatttcattaaaatgttattcctttatttaagattcacttattagtctgctgtttctctgaaaaattttagagctaggtatagtgaccgtgaacttctaacgcataatatctgtgatacagccattccgtacatgtgtgagtctgcataactttcgaactttcgaactttgttaaatgttggcactaggagtcatcagatctaggattcatcattttccagtgagaagcagagacccaaaggcctgttacttgtgcttggtcaggggactgtctgtcatgcctggaggctcttcggcacacttccccatctttcccttctgccactgtggcttcaagcacctctgttcatagagcgtctctgaaattgagtctcggtcatgacttatcccgaagtagagcaatgtgtttcctctcattgtagtttcaggactttgtcagtacaagctctgccctaggcttcttactttatactcatatcctgaaaagatgtgatttcatctatgaaggggtaaaatattggtttgtatttaattgtttgaaataaaagtgatccctataaaaaaaaaaaaaaaaaaaaaaaaaaaaaaaaaaaaaaaaaaaaaaaaaaaaaaaaaaaaaaaa

SEQ ID NO: 4 - protein, 1273 aa, Homo sapiens
MPCFYLRSCGSLLPELKLEERTEFAHRIWDTLQKLGAVYDVSHYNALLKVYLQNEYKFSPTDFLAKMEEANIQPNRVTYQRLIASYCNVGDIEGASKILGFMKTKDLPVTEAVFSALVTGHARAGDMENAENILTVMRDAGIEPGPDTYLALLNAYAEKGDIDHVKQTLEKVEKFELHLMDRDLLQIIFSFSKAGYLSMSQKFWKKFTCERRYIPDAMNLILLLVTEKLEDVALQILLACPVSKEDGPSVFGSFFLQHCVTMNTPVEKLTDYCKKLKEVQMHSFPLQFTLHCALLANKTDLAKALMKAVKEEGFPIRPHYFWPLLVGRRKEKNVQGIIEILKGMQELGVHPDQETYTDYVIPCFDSVNSARAILQENGCLSDSDMFSQAGLRSEAANGNLDFVLSFLKSNTLPISLQSIRSSLLLGFRRSMNINVWSEITELLYKDGRYCQEPRGPTEAVGNFLYNLIDSMSDSEVQAKEEHLRQYFHQLEKMNVKIPENIYRGIRNLLESYHVPELIKDAHLLVERKNLDFQKTVQLTSSELESTLETLKAENQPIRDVLKQLILVLCSEENMQKALELKAKYESDMVTGGYAALINLCCRHDKVEDALNLKEEFDRLDSSAVLDTGNYLGLVRVLAKHGKLQDAIKILKEMKEKDVLIKDTTALSFFHMLNGAALRGEIETVKQLHEAIVTLGLAEPSTNISFPLVTVHLEKGDLSTALEVAIDCYEKYKVLPRIHDVLCKLVEKGETDLIQKAMDFVSQEQGEMVMLYDLFFAFLQTGNYKEAKKIIETPGIRARSARLQWFCDRCVANNQVETLEKLVELTQKLFECDRDQMYYNLLKLYKINGDWQRADAVWNKIQEENVIPREKTLRLLAEILREGNQEVPFDVPELWYEDEKHSLNSSSASTTEPDFQKDILIACRLNQKKGAYDIFLNAKEQNIVFNAETYSNLIKLLMSEDYFTQAMEVKAFAETHIKGFTLNDAANSRLIITQVRRDYLKEAVTTLKTVLDQQQTPSRLAVTRVIQALAMKGDVENIEVVQKMLNGLEDSIGLSKMVFINNIALAQIKNNNIDAAIENIENMLTSENKVIEPQYFGLAYLFRKVIEEQLEPAVEKISIMAERLANQFAIYKPVTDFFLQLVDAGKVDDARALLQRCGAIAEQTPILLLFLLRNSRKQGKASTVKSVLELIPELNEKEEAYNSLMKSYVSEKDVTSAKALYEHLTAKNTKLDDLFLKRYASLLKYAGEPVPFIEPPESFEFYAQQLRKLRENSS

SEQ ID NO: 5 - DNA, 2202 bp, Homo sapiens
cacgcttgccgccgccccgcagaaatgcttcggttacccacagtctttcgccagatgagaccggtgtccagggtactggctcctcatctcactcgggcttatgccaaagatgtaaaatttggtgcagatgcccgagccttaatgcttcaaggtgtagaccttttagccgatgctgtggccgttacaatggggccaaagggaagaacagtgattattgagcagggttggggaagtcccaaagtaacaaaagatggtgtgactgttgcaaagtcaattgacttaaaagataaatacaagaacattggagctaaacttgttcaagatgttgccaataacacaaatgaagaagctggggatggcactaccactgctactgtactggcacgctctatagccaaggaaggcttcgagaagattagcaaaggtgctaatccagtggaaatcaggagaggtgtgatgttagctgttgatgctgtaattgctgaacttaaaaagcagtctaaacctgtgaccacccctgaagaaattgcacaggttgctacgatttctgcaaacggagacaaagaaattggcaatatcatctctgatgcaatgaaaaaagttggaagaaagggtgtcatcacagtaaaggatggaaaaacactgaatgatgaattagaaattattgaaggcatgaagtttgatcgaggctatatttctccatactttattaatacatcaaaaggtcagaaatgtgaattccaggatgcctatgttctgttgagtgaaaagaaaatttctagtatccagtccattgtacctgctcttgaaattgccaatgctcaccgtaagcctttggtcataatcgctgaagatgttgatggagaagctctaagtacactcgtcttgaataggctaaaggttggtcttcaggttgtggcagtcaaggctccagggtttggtgacaatagaaagaaccagcttaaagatatggctattgctactggtggtgcagtgtttggagaagagggattgaccctgaatcttgaagacgttcagcctcatgacttaggaaaagttggagaggtcattgtgaccaaagacgatgccatgctcttaaaaggaaaaggtgacaaggctcaaattgaaaaacgtattcaagaaatcattgagcagttagatgtcacaactagtgaatatgaaaaggaaaaactgaatgaacggcttgcaaaactttcagatggagtggctgtgctgaaggttggtgggacaagtgatgttgaagtgaatgaaaagaaagacagagttacagatgcccttaatgctacaagagctgctgttgaagaaggcattgttttgggagggggttgtgccctccttcgatgcattccagccttggactcattgactccagctaatgaagatcaaaaaattggtatagaaattattaaaagaacactcaaaattccagcaatgaccattgctaagaatgcaggtgttgaaggatctttgatagttgagaaaattatgcaaagttcctcagaagttggttatgatgctatggctggagattttgtgaatatggtggaaaaaggaatcattgacccaacaaaggttgtgagaactgctttattggatgctgctggtgtggcctctctgttaactacagcagaagttgtagtcacagaaattcctaaagaagagaaggaccctggaatgggtgcaatgggtggaatgggaggtggtatgggaggtggcatgttctaactcctagactagtgctttacctttattaatgaactgtgacaggaagcccaaggcagtgttcctcaccaataacttcagagaagtcagttggagaaaatgaagaaaaaggctggctgaaaatcactataaccatcagttactggtttcagttgacaaaatatataatggtttactgctgtcattgtccatgcctacagataatttattttgtatttttgaataaaaaacatttgtacattcctgatactgggtacaagagccatgtaccagtgtactgctttcaacttaaatcactgaggcatttttactactattctgttaaaatcaggattttagtgcttgccaccaccagatgagaagttaagcagcctttctgtggagagtgagaataattgtgtacaaagtagagaagtatccaattatgtgacaacctttgtgtaataaaaatttgtttaa

SEQ ID NO: 6 - protein, 573 aa, Homo sapiens
MLRLPTVFRQMRPVSRVLAPHLTRAYAKDVKFGADARALMLQGVDLLADAVAVTMGPKGRTVIIEQGWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 7 - DNA, 3925 bp, Homo sapiens
acagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcaagatgaagctctccctggtggccgcgatgctgctgctgctcagcgcggcgcgggccgaggaggaggacaagaaggaggacgtgggcacggtggtcggcatcgacctggggaccacctactcctgcgtcggcgtgttcaagaacggccgcgtggagatcatcgccaacgatcagggcaaccgcatcacgccgtcctatgtcgccttcactcctgaaggggaacgtctgattggcgatgccgccaagaaccagctcacctccaaccccgagaacacggtctttgacgccaagcggctcatcggccgcacgtggaatgacccgtctgtgcagcaggacatcaagttcttgccgttcaaggtggttgaaaagaaaactaaaccatacattcaagttgatattggaggtgggcaaacaaagacatttgctcctgaagaaatttctgccatggttctcactaaaatgaaagaaaccgctgaggcttatttgggaaagaaggttacccatgcagttgttactgtaccagcctattttaatgatgcccaacgccaagcaaccaaagacgctggaactattgctggcctaaatgttatgaggatcatcaacgagcctacggcagctgctattgcttatggcctggataagagggagggggagaagaacatcctggtgtttgacctgggtggcggaaccttcgatgtgtctcttctcaccattgacaatggtgtcttcgaagttgtggccactaatggagatactcatctgggtggagaagactttgaccagcgtgtcatggaacacttcatcaaactgtacaaaaagaagacgggcaaagatgtcaggaaagacaatagagctgtgcagaaactccggcgcgaggtagaaaaggccaaacgggccctgtcttctcagcatcaagcaagaattgaaattgagtccttctatgaaggagaagacttttctgagaccctgactcgggccaaatttgaagagctcaacatggatctgttccggtctactatgaagcccgtccagaaagtgttggaagattctgatttgaagaagtctgatattgatgaaattgttcttgttggtggctcgactcgaattccaaagattcagcaactggttaaagagttcttcaatggcaaggaaccatcccgtggcataaacccagatgaagctgtagcgtatggtgctgctgtccaggctggtgtgctctctggtgatcaagatacaggtgacctggtactgcttgatgtatgtccccttacacttggtattgaaactgtgggaggtgtcatgaccaaactgattccaaggaacacagtggtgcctaccaagaagtctcagatcttttctacagcttctgataatcaaccaactgttacaatcaaggtctatgaaggtgaaagacccctgacaaaagacaatcatcttctgggtacatttgatctgactggaattcctcctgctcctcgtggggtcccacagattgaagtcacctttgagatagatgtgaatggtattcttcgagtgacagctgaagacaagggtacagggaacaaaaataagatcacaatcaccaatgaccagaatcgcctgacacctgaagaaatcgaaaggatggttaatgatgctgagaagtttgctgaggaagacaaaaagctcaaggagcgcattgatactagaaatgagttggaaagctatgcctattctctaaagaatcagattggagataaagaaaagctgggaggtaaactttcctctgaagataaggagaccatggaaaaagctgtagaagaaaagattgaatggctggaaagccaccaagatgctgacattgaagacttcaaagctaagaagaaggaactggaagaaattgttcaaccaattatcagcaaactctatggaagtgcaggccctcccccaactggtgaagaggatacagcagaaaaagatgagttgtagacactgatctgctagtgctgtaatattgtaaatactggactcaggaacttttgttaggaaaaaattgaaagaacttaagtctcgaatgtaattggaatcttcacctcagagtggagttgaaactgctatagcctaagcggctgtttactgcttttcattagcagttgctcacatgtctttgggtgggggggagaagaagaattggccatcttaaaaagcaggtaaaaaacctgggttagggtgtgtgttcaccttcaaaatgttctatttaacaactgggtcatgtgcatctggtgtaggaagttttttctaccataagtgacaccaataaatgtttgttatttacactggtctaatgtttgtgagaagcttctaattagatcaattacttattttaggaaatttaagactagatactcgtgtgtggggtgaggggagggagtatttggtatgttgggataaggaaacacttctatttaatgcttccagggattttttttttttttttttaaccctcctgggcccaagtgatccttccacctcagtctcccagctaattgagaccacaggcttgttaccaccatgctcggcttttgcattaatctaagaaaaggggagagaagttaatccacatctttactcaggcaaggggcatttcacagtgcccaagagtggggttttcttgaacatacttggtttcctatttccccttatctttctaaaactgcctttctggtggctttttttaaaattattactaatgatgcttttatagctgcttggattctctgagaaatgatggggagtgagtgatcactggtattaactttatacacttggatttcatttgtaactttaggatgtaaaggtatattgtgaaccctagctgtgtcagaatctccatccctgaaatttctcattagtggtactggggtgggatcttggatggtgacattgaaactacactaaatcccctcactatgaatgggttgttaaaggcaatggtttgtgtcaaaactggtttaggattacttagattgtgttcctgaagaaaagagtccaggtaaatggtatgatcaataaaggacaggctggtgctaacataaaatccaatattgtaatcctagcactttgggaggccaaggcgggtggatcacaaggtcaagagatagagaccatctttgccaacatggtgaaactccatctctactgaaaatacaaaaattagctgggcgtggtagtgcaagctgaaggctgaggcaggagaatcactcgaacccgggaggcagaggttgcagtgagccgagatcacaccactgtactccagcccggcactccagcctggcgacaagagtgagactccacctcaaaaaaaaaaaaaagaatccaatactgcccaaggataggtattttatagatgggcaactggctgaaaggttaattctctagggctagtagaactggatcccaacaccaaactcttaattagacctaggcctcagctgcactgcccgaaaagcatttgggcagaccctgagcagaatactggtctcaggccaagcccaatacagccattaaagatgacctacagtgctgtgtaccctggggcaatagggttaaatggtagttagcaactagggctagtcttcccttacctcaaaggctctcactaccgtggaccacctagtctgtaactctttctgaggagctgttactgaatattaaaaagatagacttcaaaaaaaaaaaaaaaaaa

SEQ ID NO: 8 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 9 - DNA, 2349 bp, Homo sapiens
gacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcgtcttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcccagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaggagacagcagaagacacaacagaagacacagagcaagacgaagatgaagaaatggatgtgggaacagatgaagaagaagaaacagcaaaggaatctacagctgaaaaagatgaattctaa

SEQ ID NO: 10 - protein, 782 aa, Homo sapiens
DDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSSSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFIPDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEF

(19) United States
(12) Patent Application Publication
(10) Pub. No.: US 2005/0123571 A1
(43) Pub. Date: Jun. 9, 2005

(54) Modulation of apoptosis

(76) Inventors: Aldo A. Rossini, Sudbury, MA (US); Rita Bortell, Shirley, MA (US); Dale L. Greiner, Hubbardston, MA (US); John P. Mordes, Waban, MA (US); Silvia Corvera, Westborough, MA (US); Leanne Wilson-Fritch, Auburn, MA (US); Malini Pandarpurkar, Farmington Hills, MI (US); Helle Markholst, Charlottenlund (DK); Lars Hornum, Kirke Eskilstrup (DK)

Correspondence Address: LAHIVE & COCKFIELD, LLP., 28 STATE STREET, BOSTON, MA, 02109, US

(21) Appl. No.: 10/863,012

(22) Filed: Jun. 7, 2004

Related U.S. Application Data
(60) Provisional application No. 60/476,550, filed on Jun. 6, 2003.
(60) Provisional application No. 60/478,234, filed on Jun. 13, 2003.

(51) Int. Cl. C12Q 1/68; C07H 21/04; C12N 9/64; A61K 39/00

(52) U.S. Cl. 424/277.1; 435/6; 435/69.1; 435/226; 435/320.1; 435/325; 536/23.2

(57) ABSTRACT

The present invention relates, at least in part, to methods of modulating apoptosis in cells, comprising contacting the cell with a compound that modulates the expression, post-translational modification, or activity of Ian4, leucine-rich protein of 130 kD, GRP78, GRP94, or hsp60. The subject methods can be used to treat a variety of different disorders, for example, autoimmune disease and neoplasia. The invention also pertains to methods for identifying compounds that modulates the activity of Ian-4, leucine-rich protein of 130 kD, GRP78, GRP94, or hsp60.

### RELATED APPLICATIONS

This application claims priority to copending U.S. Provisional Applications Nos. 60/476,550 and 60/478,234, filed on Jun. 6, 2003 and Jun. 13, 2003 respectively, the contents of which are incorporated herein by reference.

### GOVERNMENT FUNDING

This invention was made, at least in part, with government support under grant nos. DK49106, DK36024, DK25306, DK60837-01A1, and DK32520 awarded by the National Institutes of Health. The government may, therefore, have certain rights in this invention.

### BACKGROUND OF THE INVENTION

Apoptosis has been implicated in controlling the amount and distribution of certain differentiated cell types, such as cells of the hematopoietic lineage, as well as other somatic and germ cells. Apoptosis was first described as a morphologic pattern of cell death characterized by cell shrinkage, membrane blebbing and chromatin condensation culminating in cell fragmentation (Kerr et al., 1972. Br. J. Cancer 26:239). Cells undergoing apoptosis display a characteristic pattern of internucleosomal DNA cleavage (Wyllie. 1980. Int. Rev. Cytol. 69:251; Abrams et al. 1993. Development. 117:29).

Apoptosis is mainly controlled by the activation of the caspase (aspartate-specific cysteine protease) cascade. Caspases can be activated by a pathway involving mitochondria (the receptor-independent or intrinsic pathway) or by cognate interactions of death receptors with their ligands (the extrinsic pathway). The mitochondrial pathway is controlled by pro- and anti-apoptotic members of the Bcl-2 family of proteins. Signals that induce cell stress causes translocation of pro-apoptotic Bcl-2 family proteins from the cytosol to the mitochondria where they induce the release of cytochrome c and other apoptogenic factors (e.g. procaspase-2, procaspase-3, procaspase-8, procaspase-9, Smac/Diablo, AIF, or endonuclease G) from the interior of the mitochondria. Anti-apoptitic Bcl-2 proteins work to prevent the release of apoptogenic factors. Cytochrome c acts to initiate the caspase activation cascade by causing the oligomerization of apoptotic protease activating factor (APAF). This pathway results in the activation of procaspase-9, which then activates procaspase-3. Alternatively, ligation of cell death receptors results in the activation of procaspase-8 by the extrinsic pathway. The end result of activation of either of these pathways is cleavage of cellular substrates and the morphological and biochemical changes characteristic of apoptosis.

In addition to playing a role in normal development, apoptosis has been implicated in pathologic conditions, such as Alzheimer's disease, Parkinson's disease, amyotrophic lateral sclerosis (ALS), spinal muscular atrophy, and autoimmune disorders (see e.g., Passer et al. J Biol Chem 1999 Aug. 20; 274: 24007). Moreover, the ability to modulate apoptosis in cells would be valuable in controlling undesirable cell proliferation, e.g., the proliferation of cancer cells. Thus, the identification of molecules involved in apoptotic pathways and agents that can modulate these molecules will be useful in controlling cell proliferation, differentiation, and/or apoptosis in numerous applications. Specifically, the identification of molecules involved in apoptosis of lymphoid cells will be of value in treating a variety of immunological disorders.

### SUMMARY OF THE INVENTION

The present invention is based, at least in part, on the discovery that members of Ian gene family function to prevent apoptosis by regulating mitochondrial integrity in cells of lymphoid origin. As shown in the examples, the absence of the Ian family member Ian4 in T cells, but not thymocytes or B cells, causes mitochondrial dysfunction and spontaneous apoptosis, establishing Ian4 as a novel, tissue-specific regulator of the receptor-independent caspase pathway in these cells. In addition, the absence of functional Ian4 results in increased mitochondrial levels of stress-inducible chaperones and a novel leucine-rich protein of 130 kD. T cell activation and caspase 8 inhibition both prevented apoptosis, whereas transfection of T cells with Ian4-specific siRNA recapitulated the apoptotic phenotype. These data identify Ian4 as a new mitochondrial regulator of caspase-dependent T cell apoptosis. Furthermore, hsp60, leucine rich protein of 130 kD, GRP78, and GRP94 have been implicated in apoptosis. Therefore, agents that modulate these molecules are useful in regulating apoptosis.

Accordingly, in one aspect, the present invention provides a method of specifically modulating apoptosis in a cell of lymphoid origin, comprising contacting the cell with a compound that modulates the expression or activity of an Ian-related gene or protein, such that apoptosis in the cell is specifically modulated.

In one embodiment, the Ian-related gene or protein is Ian4.

In one embodiment, the cell is a CD4+ cell e.g., a CD4+ T cell. In another embodiment, the cell is a CD8+ cell, e.g., a CD8+ T cell.

In one embodiment, apoptosis is downmodulated. In another embodiment, apoptosis is upmodulated.

In one embodiment, the cell has previously received a signal that induces apoptosis.

In one embodiment, the compound modulates mitochondrial inner membrane permeabilization.

In another embodiment, the compound modulates the release of an apoptogenic factor from mitochondria within the cell. In a preferred embodiment, the apoptogenic factor is procaspase-2, procaspase-3, procaspase-8, procaspase-9, cytochrome-c, Smac/Diablo, AIF, or endonuclease G.

In another aspect, the invention is directed to a method of treating an autoimmune disease, comprising contacting a cell with a compound that modulates the expression or activity of an Ian gene or protein, such that apoptosis is modulated and the autoimmune disease is treated.

In one embodiment, the autoimmune disease is diabetes.

In another embodiment, the Ian gene or protein is Ian4.

In another embodiment, the expression or activity of Ian4 is upmodulated.

In one embodiment, the compound modulates mitochondrial membrane permeabilization. In a preferred embodiment, the compound modulates mitochondrial inner membrane permeabilization.

In another embodiment, the compound modulates the release of an apoptogenic factor from mitochondria within the cell. In a preferred embodiment, the apoptogenic factor is procaspase-2, procaspase-3, procaspase-8, procaspase-9, cytochrome-c, Smac/Diablo, AIF, or endonuclease G.

In still another aspect, the invention pertains to a method of treating neoplasia, comprising contacting a cell, e.g., a cell of lymphoid origin, with a compound that modulates the expression or activity of an Ian-related gene or protein, such that apoptosis is modulated and neoplasia is treated.

In one embodiment, the Ian gene or protein is Ian 4. In another embodiment the expression or activity of Ian 4 is upmodulated. In another embodiment, the expression or activity of Ian4 is downmodulated.

In one embodiment, the compound modulates mitochondrial membrane permeabilization. In a preferred embodiment, the compound modulates mitochondrial inner membrane permeabilization.

In another embodiment, the compound modulates the release of an apoptogenic factor from mitochondria within the cell. In a preferred embodiment, the apoptogenic factor is procaspase-2, procaspase-3, procaspase-8, procaspase-9, cytochrome-c, Smac/Diablo, AIF, or endonuclease G.

In another aspect, the invention pertains to a method of modulating receptor-independent apoptosis, comprising contacting a cell, e.g., a cell of lymphoid origin, with a compound that modulates the expression or activity of caspase 8, such that receptor-independent apoptosis is modulated.

In another embodiment, the cell is a T cell.

In yet another aspect, the invention pertains to a method of treating an autoimmune disease, comprising contacting a cell, e.g., a cell of lymphoid origin, with a compound that modulates the expression or activity of caspase 8, such that receptor-independent apoptosis is modulated and the autoimmune disease is treated.

In another aspect, the invention pertains to a method of modulating apoptosis, comprising contacting a cell, e.g., a cell of lymphoid origin, with a compound that modulates the expression or activity of the leucine-rich protein of 130 kD, such that apoptosis is modulated.

In still another aspect, the invention pertains to a method of modulating apoptosis, comprising contacting a cell, e.g., a cell of lymphoid origin, with a compound that modulates the expression or activity of hsp60, GRP78 or GRP94, such that apoptosis is modulated.

In yet another aspect, the invention pertains to a method of treating an autoimmune disease, comprising contacting a cell, e.g., a cell of lymphoid origin, with a compound that modulates the expression or activity of leucine rich protein of 130 kD, hsp60, GRP78 or GRP94, such that apoptosis is modulated and the autoimmune disease is treated.

In another aspect, the invention pertains to a method of treating neoplasia, comprising contacting a cell, e.g., a cell of lymphoid origin, with a compound that modulates the expression or activity of leucine rich protein of 130 kD, hsp60, GRP78 or GRP94, such that apoptosis is modulated and neoplasia is treated.

In still another aspect, the invention pertains to a method for identifying a compound that modulates the anti-apoptotic activity of an Ian gene or protein, comprising:
contacting a cell expressing a functional or nonfunctional Ian gene or protein with a test compound and;
determining the ability of the test compound to modulate the expression or anti-apoptotic activity of the Ian gene or protein to thereby identify a compound that modulates the anti-apoptotic activity of the Ian gene or protein.

In one embodiment, the Ian gene or protein is Ian4.

In one embodiment, the method further comprises subjecting the cell expressing the Ian gene or protein to a signal that induces apoptosis.

In one embodiment, the ability of the test compound to modulate the anti-apoptotic activity of an Ian gene or protein is determined by measuring apoptosis in the cell.

In another embodiment, the ability of the test compound to modulate the expression or anti-apoptotic activity of the Ian gene or protein is determined by measuring a change in the membrane permeabilization of mitochondrial membranes in the cell. In a preferred embodiment, the inner mitochondrial membrane permeabilizaton is measured.

In still another embodiment, the membrane permeabilization of mitochondrial inner membranes is measured by determining mitochondrial inner membrane potential.

In another embodiment, the compound modulates the release of an apoptogenic factor from mitochondria within the cell. In a preferred embodiment, the apoptogenic factor is procaspase-2, procaspase-3, procaspase-8, procaspase-9, cytochrome-c, Smac/Diablo, AIF, or endonuclease G.

In yet another embodiment, the cell is engineered to express a heterologous nucleic acid molecule encoding a functional or non-functional Ian protein.

In still another aspect, the invention pertains to a method of identifying compounds which modulate apoptosis in a cell of lymphoid origin, comprising:
contacting a cell which expresses a functional or non-functional form of an Ian gene or protein with a compound;
determining the effect of the compound on apoptosis, to thereby identify a compound that modulates apoptosis in the cell.

In one embodiment, the Ian protein is Ian4.

In another embodiment, the method further comprises subjecting the cell to a signal that induces apoptosis.

In one embodiment, apoptosis is increased. In another embodiment, apoptosis is decreased.

In one embodiment, the ability of the test compound to modulate apoptosis is determined by measuring apoptosis in the cell.

In another embodiment, the ability of the test compound to modulate apoptosis is determined by measuring a change in the membrane permeabilization of mitochondrial membranes in the cell. In a preferred embodiment, the membrane permeabilization of the inner mitochondrial membrane is measured.

In still another embodiment, the membrane permeabilization of mitochondrial inner membranes is measured by determining mitochondrial inner membrane potential.

In another embodiment, the ability of the test compound to modulate apoptosis within the cell is measured by the release of an apoptogenic factor from mitochondria within the cell. In a preferred embodiment, the apoptogenic factor is procaspase-2, procaspase-3, procaspase-8, procaspase-9, cytochrome-c, Smac/Diablo, AIF, or endonuclease G.

In one embodiment, the ability of the test compound to modulate apoptosis is determined by measuring the expression or activity of a component of the receptor-independent apoptotic pathway.

In another embodiment, the ability of the test compound to modulate apoptosis is determined by measuring the expression or activity of the leucine-rich protein of 130 kD, hsp60, GRP78 or GRP94.

In still another aspect, the invention pertains to a method for identifying a compound that modulates the receptor-independent pro-apoptotic activity of caspase 8, comprising:
contacting a cell expressing caspase 8 with a test compound and;
determining the ability of the test compound to modulate the receptor-independent pro-apoptotic activity of caspase 8 to thereby identify a compound that modulates the receptor-independent pro-apoptotic activity of caspase 8.

In one embodiment, the ability of the test compound to modulate the receptor-independent pro-apoptotic activity of caspase 8 is determined by measuring apoptosis in the cell.

In another embodiment, the ability of the test compound to modulate the receptor-independent pro-apoptotic activity of caspase 8 is determined by measuring a change in the membrane permeabilization of mitochondrial membranes in the cell. In a preferred embodiment, the permeabilization of the inner mitochondrial membrane is measured.

In one embodiment, the membrane permeabilization of mitochondrial inner membranes is measured by determining mitochondrial inner membrane potential.

In another embodiment, the ability of the test compound to modulate apoptosis is determined by measuring the release of an apoptogenic factor from mitochondria within the cell.

In one embodiment, the apoptogenic factor is procaspase-2, procaspase-3, procaspase-8, procaspase-9, cytochrome-c, Smac/Diablo, AIF, or endonuclease G.

In one embodiment, the ability of the test compound to modulate apoptosis is determined by measuring the expression or activity of a component of the recertor-independent apoptotic pathway.

In another embodiment, the ability of the test compound to modulate apoptosis is determined by measuring the expression or activity of the leucine rich protein of 130 kD, hsp60, GRP78 or GRP94.

In still another aspect, the invention provides a method for identifying a compound that modulates the expression or anti-apoptotic activity of an Ian gene or protein, comprising:
administering a test compound to a model organism with a mutant Ian phenotype;
determining the ability of the test compound to rescue the mutant Ian phenotype to thereby identify a compound that modulates the expression or anti-apoptotic activity of the Ian gene or protein.

In one embodiment, the Ian gene or protein is Ian4.

In another embodiment, the organism is further subjected to a signal that induces apoptosis.

In still another aspect, the invention provides a method of identifying a compound that modulates apoptosis in a cell of lymphoid origin, comprising:
administering a test compound to an animal model with a mutant Ian phenotype; determining the ability of the test compound to rescue the mutant Ian phenotype, to thereby identify a compound that modulates apoptosis in the cell.

In one embodiment, the Ian protein is Ian4.

In another embodiment, the organism is further subjected to a signal that induces apoptosis.

In still another aspect, the invention provides a method for identifying a compound capable of treating an autoimmune disorder characterized by lymphopenia, comprising:
administering a test compound to a animal model with a mutant Ian phenotype; determining the ability of the test compound to rescue the mutant Ian phenotype, to thereby identify a compound capable of treating a disorder characterized by lymphopenia.

In one embodiment, the Ian protein is Ian4.

In another embodiment, the organism is further subjected to a signal that induces apoptosis.

In another embodiment, the disorder is diabetes.

In still another aspect, the invention provides for identifying a compound capable of treating a neoplastic disorder of lymphoid origin characterized by aberrant Ian expression, comprising:
administering a test compound to a non-human transgenic organism capable of overexpressing an Ian protein; determining the ability of the test compound to rescue the mutant Ian phenotype, to thereby identify a compound capable of treating a neoplastic disorder of lymphoid origin.

In one embodiment, the Ian protein is Ian4.

In another embodiment, the organism is further subjected to a signal that induces apoptosis.

In another embodiment, the neoplastic disorder is leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burkett's lymphoma.

In still another aspect, the invention provides a method for determining if a subject is at risk for an autoimmune disorder characterized by lymphopenia comprising detecting, in a sample of cells from the subject, the presence or absence of a genetic lesion, wherein the genetic lesion is characterized by an alteration affecting the misexpression of the Ian gene.

In one embodiment, the Ian protein is Ian4.

In another embodiment, the disorder is diabetes.

In still another aspect, the invention provides a method for determining if a subject is at risk for a neoplastic disorder characterized by Ian overexpression comprising detecting, in a sample of cells from the subject, the presence or absence of a genetic lesion, wherein the genetic lesion is characterized by an alteration affecting the integrity of a gene encoding an Ian protein or overexpression of the Ian gene.

In one embodiment, the Ian protein is Ian4.

In another embodiment, the disorder is leukemia, acute lymphocytic leukemia, acute lymphoblastic leukemia, B-cell lymphoma, T-cell lymphoma, Hodgkins lymphoma, non-Hodgkins lymphoma, hairy cell lymphoma or Burkett's lymphoma.

### BRIEF DESCRIPTION OF THE DRAWINGS

FIG. 1 shows western analyses of Ian4 protein. Panel A shows thymocyte (Thy) and lymph node cell (LNC) lysates from Ian4^{+/+} BBDR (“DR”) and WF and Ian4^{-/-} BBDP (“DP”) rats. Panel B shows lymph node T cell, splenic B cell, and macrophage lysates from Ian4^{+/+} BBDR and WF.ART2a²¹ rats. Panel C shows purified mitochondrial lysates from T cells and thymocytes of Ian4^{+/+} BBDR and WF and Ian4^{-/-} BBDP rats. Actin and cytochrome C were used as loading controls.

FIG. 2 shows mitochondrial membrane potential (Δψₘ). Panel A shows Δψₘ of thymocytes from Ian4^{+/+} (WF, BBDR) and Ian4^{-/-} (BBDP) rats; shown are the relative percentages of cells fluorescing red (vertical axis, high Δψₘ) and green (horizontal axis, low Δψₘ). The majority of thymocytes from both Ian4^{+/+} and Ian4^{-/-} rats display high Δψₘ. Panel B shows Δψₘ of lymph node T cells. Horizontal bars in histograms indicate gates used to identify αβTCR⁺ cells. The T lymphopenia of BBDP rats is evident. The dot plots show that the percentage of T cells with high Δψₘ was lower in Ian4^{-/-} than in Ian4^{+/+} rats.

FIG. 3 shows kinetics of Δψₘ and DNA fragmentation. Panel A shows peripheral T lymphocytes from Ian4^{+/+} (WF and BBDR) rats and Ian4^{-/-} (BBDP) rats were assayed for Δψₘ at time of isolation (Time Zero) and after 17 hrs of culture. Panel B shows the percentage of cells with sub-diploid DNA. Panels C and D show Ian4^{-/-} BBDP T cells that were incubated for 17 hrs with anti-CD3 plus anti-CD28 mAbs (C) or for 30 min with cyclosporin A (D).

FIG. 4 shows siRNA and proteomic analyses. Panel A shows transfection of Ian4-specific siRNA increased T cell apoptosis. Bars depict the percentage of cells with sub-diploid DNA after 48 hr of treatment. Panel B shows purified mitochondrial proteins from Ian4^{+/+} WF and Ian4^{-/-} BBDP rat T cells resolved by SDS-PAGE. Bands 1, 2, and 5 were differentially expressed in Ian4^{+/+} vs. Ian4^{-/-} T cells, excised, and identified by MALDI-TOF mass spectrometry.

FIG. 5 shows consensus GTPase domains in Ian4 proteins.

### DETAILED DESCRIPTION OF THE INVENTION

The present invention is based, at least in part, on the discovery that members of the Ian gene family function to prevent apoptosis in cells of lymphoid origin. The inventors herein disclose that the absence of the Ian gene family member, Ian4, in T cells causes tissue specific mitochondrial dysfunction and spontaneous apoptosis. In addition, the absence of functional Ian4 results in increased mitochondrial levels of stress-inducible chaperones, hsp60, GRP78, and GRP94 and a novel leucine-rich protein of 130 kD. Apoptosis could be prevented by T cell activation or with inhibitors of caspase 3 or caspase 8. The role of Ian4 in apoptosis was confirmed by showing that transfection of T cells with Ian4-specific siRNA recapitulated the apoptotic phenotype. These data identify Ian proteins as a new, tissue-specific mitochondrial regulators of the receptor-independent apoptotic caspase pathway. Accordingly, the invention provides, inter alia, methods of identifying modulators of Ian proteins, and methods of modulating Ian activity in a cell. In addition, the invention provides for modulation of stress-inducible chaperones, hsp60, GRP78, GRP94, and/or modulation of a leucine rich protein of 130 kD which have also been implicated in modulation of apoptosis and for methods of identifying modulators of these molecules.

These and other aspects of the invention are described in further detail in the following subsections:

I. Definitions

The nucleotide and amino acid sequences of several “Ian” or Immune-associated nucleotide gene family members are known in the art and include at least 10 human genes (hIan1-10) and at least 11 mouse Ian genes (mIan1-11) (Krucken et al. 1999, J. Biol. Chem, 274: 24383; Poirier et al., 1999, J. Immunol., 163: 4960; Daheron et al., 2001, Nucleic Acids Res., 29: 1308). In humans, all known Ian gene family members are located in a 300-kb within the 7q36.1 chromosomal region (MacMurray et al, 2002, Genome Res., 12: 1209; Cambot et al, 2002, Blood, 99:3293; Stamm et al, 2002, Gene, 282: 159). Ian family members are anti-apoptotic factors characterized by the presence of a GTP-binding domain, as well as a carboxyterminal transmembrane domain that localizes the protein to the outer mitochondrial membrane. These proteins are also characterized by prominent expression in in lymphoid tissues. “Ian4” as used herein includes all nucleotide and amino acid sequences from all mammalian species. In humans, Ian4 has also been referred to as Ian411 and Ian5. The nucleotide and amino acids sequences of human Ian4 are set forth in SEQ ID NO:1 and SEQ ID NO:2 respectively. In addition, human Ian4 is found at GenBank accession no: NP_{—}060854 (gi:28416949) or NM_{—}018384 (gi:28416948).

Ian4 is well conserved among species and several othologues of the human Ian4 have been described. cDNA sequences encoding the mouse and rat Ian4 orthologues can be found at GenBank. AB164418; gi:46093643 and GenBank AAL17698 gi:21591786 respectively. An alignment of human, mouse and rat Ian4 molecules is shown, e.g., in Hornum et al. 2002. diabetes. 51:1972.

As used herein, the term “activity” of an Ian family member, e.g, Ian4, includes its ability to modulate apoptosis in a cell, in particular, a cell of lymphoid origin, such as a T lymphocyte. Specifically, Ian4 acts as an anti-apoptotic factor in T cells.

As used herein; the term “modulate Ian, e.g., Ian4, activity or expression” includes upregulation and downregulation of Ian activity, e.g., anti-apoptotic activity, or Ian expression (e.g., at the level of transcription or translation) in a cell.

As used herein the term “Caspase 8” refers to the active form of caspase 8 that results from cleavage of procaspase 8. As used herein, the term “modulate caspase 8 activity or expression” includes upregulation and downregulation of caspase 8 activity, e.g., pro-apoptotic activity, expression (e.g., at the level of transcription or translation), or post-transcriptional modification (e.g., cleavage into its active form) in a cell. The nucleotide and the amino acid sequence of various forms of caspase 8 are known in the art, see, e.g., GenBank accession numbers: NM_{—}033357 (gi:15718709, variant D); NM_{—}033356 (gi:15718707, variant C); NM_{—}001228 (gi:9961351, variant A); NM_{—}033358 (gi:15718711, variant E); and NM_{—}033355 (gi:15718705, variant B). Other art recognized caspases are also within the scope of the invention.

As used herein the term “leucine rich protein of 130 kD” refers to the leucine rich protein of 130 kD that was previously referred to as gp130. The nucleotide and amino acid sequence of the human 130 kD leucine rich protein is known in the art, see e.g., Hou, J. et al. 1994. In Vitro Cell. Dev. Biol. 30A:111; GenBank accession no. S27954 (gi:284289) NP_{—}573566 (gi:18959202) and are shown in SEQ ID NO:3 and SEQ ID No:4 respectively.

As used herein the terms hsp60, GRP78, and GRP94 refer to stress-inducible chaperones. The nucleic acid and amino acid sequences of these molecules are known in the art. For example, the nucleotide and amino acid sequence of human hsp60 can be found in Jindal et al. (1989. Mol. Cell. Biol. 9:2279) or in GenBank Accession No: NM 002156 (gi: 4504520) and are shown in SEQ ID NO:5 and 6 respectively. The nucleotide and amino acid sequence of human GRP78 can be found in Ting and Lee. 1988. DNA 7:275 or at GenBank Accession No: NM_{—}005347 (gi21361242) and are shown in SEQ ID NO:7 and 8 respectively. The nucleotide and amino acid sequence of human GRP94 can be found in GenBank Accession no. AY040226 (gi: 15010549) or AAK74072 (gi: 15010550) and are shown in SEQ ID NO:9 and 10 respectively.

As used herein the term “apoptosis” includes programmed cell death which can be characterized using techniques which are known in the art. Apoptotic cell death can be characterized directly, e.g., by cell shrinkage, membrane blebbing and chromatin condensation culminating in cell fragmentation. Cells undergoing apoptosis also display a characteristic pattern of internucleosomal DNA cleavage. Alternatively, apoptosis can be characterized indirectly by changes in the activity or expression of members of the apoptotic pathway, e.g. increased mitochondrial release of cytochrome c. As used herein, the term “modulating apoptosis” includes modulating programmed cell death in a cell of lymphoid origin, such as a T cell. As used herein, the term “modulates apoptosis” includes either upregulation or downregulation of apoptosis in a cell. Modulation of apoptosis is discussed in more detail below and can be useful in ameliorating various disorders, e.g., immunological disorders or cancer.

As used herein the term “cell of lymphoid origin” includes T and B lymphocytes or cells, as well as natural killer cells (i.e., NK cells).

As used herein the term “receptor-independent apoptosis” includes apoptosis that is not brought about by ligation of a death receptor (e.g., CD95, DR3/WSL, or (TRAIL)/APO-2L receptors) on a cell and activation of the extrinsic pathway, but rather is brought about by stress on the cell (e.g., absence of a cytokine).

As used herein the term “mature T cell” or “mature T lymphocyte” includes mature single positive cells, e.g., CD4+ T cells (cells bearing the CD4 molecule) and CD8+ T cells (cells bearing the CD8+ molecule). The term “mature T cell” does not include thymocytes or double positive (CD4+/CD8+) cells. The term T cell includes Th1 cells and Th2 cells.

As used herein, the term “non-T lymphocytes” includes other lymphocytes which are not T lymphocytes, e.g., B cells and monocytes that are not derived from the thymus and do not express T cell markers, e.g., CD3 or the T cell receptor.

As used herein, the term “mitochondrial integrity” includes the ability of mitochondria to maintain an electro chemical (proton) gradient and retain apoptogenic factors such as cytochrome c within the mitochondrial matrix or intermembrane space.

A used herein, the term “mitochondrial inner membrane permeabilization” includes an increase in mitochondrial inner membrane permeability, such that solutes are extruded through the membrane, water is influxed, and, ultimately, the transmembrane potential of the membrane is disrupted.

As used herein, the term “mitochondrial inner membrane potential” includes the electro chemical (proton) gradient that results from the electron-transport-chain-mediated pumping of protons out of the inner mitochondrial membrane.

As used herein the term “autoimmune disease” includes diseases or disorders which involve inappropriate or unwanted immune responses to self antigens.

As used herein, “heterologous DNA” or “heterologous nucleic acid” includes DNA that does not occur naturally as part of the genome in which it is present or which is found in a location or locations in the genome that differs from that in which it occurs in nature or which is operatively linked to DNA to which it is not normally linked in nature (i.e., a gene that has been operatively linked to a heterologous promoter). Heterologous DNA is not naturally occurring in that position or is not endogenous to the cell into which it is introduced, but has been obtained from another cell. Heterologous DNA can be from the same species or from a different species. In one embodiment, it is mammalian, e.g., human. DNA that one of skill in the art would recognize or consider as heterologous or foreign to the cell in which is expressed is herein encompassed by the term heterologous DNA. In contrast, the term endogenous, used with respect to nucleic acid, includes DNA that is native to the host cell, i.e., has not been introduced but is naturally-occurring in the cells or genome of an organism (i.e. not altered or introduced by techniques of molecular biology).

The terms “heterologous protein”, “recombinant protein”, and “exogenous protein” are used interchangeably throughout the specification and refer to a polypeptide which is produced by recombinant DNA techniques wherein, generally, DNA encoding the polypeptide is inserted into a suitable expression vector which is in turn used to transform a host cell to produce the heterologous protein. That is, the polypeptide is expressed from a heterologous nucleic acid molecule.

As used herein, the term “antibody” is intended to include immunoglobulin molecules and immunologically active portions of immunoglobulin molecules, i.e., molecules that contain an antigen binding site which binds (immunoreacts with) an antigen, such as Fab and F(ab′)₂ fragments, single chain antibodies, intracellular antibodies, scFv, Fd, or other fragments. Preferably, antibodies of the invention bind specifically or substantially specifically to a protein of the invention (e.g., Ian4, leucine rich protein of 130 kD, hsp60, GRP78, or GRP94). The terms “monoclonal antibodies” and “monoclonal antibody composition”, as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. Monoclonal antibody compositions thus typically display a single binding affinity for a particular antigen with which they immunoreact.

A “small molecule” includes compounds which are not the product of gene transcription or translation (protein, RNA or DNA). Preferably a “small molecule” is a low molecular weight compound, e.g., less than 7500 amu, more preferably less 5000 amu and even more preferably less than 1000 amu.

An “agent” shall include compounds such as small molecules, antibodies, peptides.

II. Modulation of Apoptosis

As shown in the instant examples, a frameshift mutation in the Ian4 gene results in severe T cell lymphopenia that is associated with mitochondrial dysfunction, increased mitochondrial levels of stress-inducible chaperones and a novel leucine-rich protein, and spontaneous T cell-specific apoptosis. Thus, the frameshift mutation is a loss-of-function mutation that results in the failure to inhibit apoptosis. T cell activation and caspase 8 inhibition both prevented apoptosis, whereas transfection of T cells with Ian4-specific siRNA caused apoptosis. These data identify Ian4 as a regulator of apoptosis. In addition, hsp60, GRP78, and GRP94 have been implicated in apoptosis. Accordingly, the invention provides for the modulation of the expression and/or activity of one or more of the proteins of the invention (Ian4, hsp60, GRP78, or GRP94) to modulate cell proliferation, viability, or apoptosis in cells of lymphoid origin. In another embodiment, the invention provides for modulation of caspase 8 activity, expression, or post-translational modification to modulate receptor-independent apoptosis in a cell of lymphoid origin. In one embodiment, expression and/or activity is modulated in a T cell. Given the key role of T cells in immune responses, the subject methods can be used to modulate normal or aberrant immune responses (e.g., immune deficiencies, autoimmune diseases or other unwanted immune responses) or to modulate the growth of immune cells, e.g., neoplasias, such as cancers, in particular, leukemia. In one embodiment, expression and/or activity of a protein of the invention is modulated in a thymocyte. In one embodiment, expression and/or activity of a protein of the invention is modulated in a mature T cell. In one embodiment, apoptosis is modulated in a single positive T cell, e.g., bearing CD4 or CD8 and not in a cell bearing both CD4 and CD8. In another embodiment, expression and/or activity of a protein of the invention is modulated in a B cell. In another embodiment, expression and/or activity of a protein of the invention is modulated in a cell that does not normally express a functional Ian4, e.g., Ian4, protein. In another embodiment, apoptosis is modulated in a cell other than a T cell. Accordingly, the subject methods can also be used to modulate normal or aberrant cell development, proliferation, or apoptosis in non-T cells.

In one embodiment, the methods of the invention modulate receptor-independent apoptosis. In another embodiment, the methods of the invention modulate receptor-dependent apoptosis.

In one embodiment, apoptosis is modulated in a cell that has previously received a signal that induces apoptosis, e.g., the cell has been subjected to a stress signal that induces receptor-independent apoptosis or a death receptor that induces apoptosis in the cell has been ligated. In another embodiment, apoptosis is modulated in a cell that has not previously received a signal that induces apoptosis.

In one embodiment, Ian 4 expression or activity (e.g., Ian4 expression or activity) is modulated. In another embodiment, the expression or activity of a polypeptide whose expression is controlled by an Ian gene family member is modulated. In a preferred embodiment, the Ian gene family member is Ian4. In another embodiment, expression or activity of one or more of leucine rich protein of 130 kD, GRP94, GRP78, and hsp60 is modulated. In another embodiment, the expression or activity of an Ian gene family member (e.g., Ian4) is modulated in combination with modulation of the expression or activity of one or more of leucine rich protein of 130 kD, GRP94, GRP78, and hsp60. In still another embodiment, caspase activity, expression, or post-translational modification is modulated in order to modulate receptor-independent apoptosis in a cell. In one embodiment, the caspase is caspase 8.

Depending upon the disorder or condition to be treated, apoptosis can be up- or downmodulated. Exemplary Ian stimulatory agents, agents that stimulate Ian expression or anti-apoptotic activity of Ian and therefore reduce or inhibit apoptosis, and Ian inhibitory agents, agents that inhibit Ian expression or anti-apoptotic activity and therefore enhance or stimulate apoptosis, are described in more detail below.

In one embodiment, agents that modulate the expression or activity of leucine rich protein of 130 kD, GRP94, GRP78, and/or hsp60 can be used to modulate apoptosis. In another embodiment, agents that stimulate caspase 8 expression, activity or post-translational processing into the active form can be used to enhance or stimulate apoptosis and agents that inhibit caspase 8 expression, activity or post-translational processing into the active form can be used to enhance or reduce or inhibit apoptosis. Such agents can be used alone, in combination, or with other agents known in the art to promote or inhibit apoptosis.

A. Stimulatory Agents

According to the methods of the invention, to stimulate the expression and/or activity of one or more of the proteins of the invention, a cell is contacted with a stimulatory agent that stimulates expression and/or activity of one or more of the proteins.

In one embodiment, the expression and/or anti-apoptotic activity of a functional Ian gene or protein is stimulated in a cell that already expresses it, but at insufficient levels, in order to decrease apoptosis.

In a more specific embodiment, to stimulate the expression and/or anti-apoptotic activity of Ian4, a cell is contacted with a stimulatory agent that stimulates expression and/or activity of Ian4.

In one embodiment, the expression and/or activity of Ian4 is increased in a cell that already expresses Ian4.

In another embodiment, the expression and/or activity of functional Ian gene or protein is stimulated in cells that do not normally express functional Ian gene or protein, such as lymphoid cells which express a mutated non-functional form of the Ian gene (e.g. a frameshift mutation) or non-lymphoid cells, in order to decrease apoptosis.

In one embodiment, apoptosis can be inhibited in cells that do not normally express functional Ian4, such as lymphoid cells which express a mutated form of Ian4 or non-lymphoid cells.

In one embodiment, the expression and/or pro-apoptotic activity of functional leucine rich protein of 130 kD, GRP94, GRP78, or hsp60, is stimulated in a cell that already expresses one or more of these molecules, but at insufficient levels, in order to increase apoptosis. In another embodiment, the expression and/or pro-apoptotic activity of one of these molecules can be stimulated in cells that do not normally express a functional form, in order to increase apoptosis.

In another embodiment, the pro-apoptotic activity of caspase 8 is stimulated in a cell that already expresses the functional molecule, in order to increase receptor-independent apoptosis. In one embodiment, receptor-independent apoptosis can be stimulated in cells that do not normally express a functional form of this molecule, in order to increase receptor-independent apoptosis.

In another embodiment, to stimulate the pro-apoptotic activity of leucine rich protein of 130 kD, GRP94, GRP78, or hsp60, a cell is contacted with a stimulatory agent that stimulates expression and/or activity of one or more of these molecules. In one embodiment, the expression or activity of leucine rich protein of 130 kD, GRP94, GRP78, or hsp60 is increased in a cell that already expresses one or more of the molecules. In one embodiment, apoptosis can be modulated in cells that do not normally express functional forms of these molecules.

In another embodiment, to stimulate the pro-apoptotic activity of caspase 8, a cell is contacted with a stimulatory agent that stimulates expression, post-translational processing and/or activity of this molecule. In one embodiment, the expression or activity of caspase 8 is increased in a cell that already expresses the molecule to increase receptor-independent apoptosis. In one embodiment, receptor-independent apoptosis can be stimulated in cells that do not normally express a functional form of this molecule.

A preferred stimulatory agent is a nucleic acid molecule encoding a functional protein of the invention, wherein the nucleic acid molecule is introduced into the cell in a form suitable for expression of the functional protein in the cell (ie. gene therapy). For example, a cDNA is cloned into a recombinant expression vector and the vector is transfected into the cell.

To express a protein in a cell, typically a cDNA is first introduced into a recombinant expression vector using standard molecular biology techniques. cDNA encoding a protein of the invention can be obtained, for example, by amplification using the polymerase chain reaction (PCR) or by screening an appropriate cDNA library. The nucleotide sequences of cDNAs known in the art can be used for the design of PCR primers that allow for amplification of a cDNA by standard PCR methods or for the design of a hybridization probe that can be used to screen a cDNA library using standard hybridization methods. For example, Ian4 cDNAs from other mammalian species can be isolated using standard molecular biology techniques (e.g., PCR or cDNA library screening) and primers or probes designed based upon the known sequences.

In addition, variant forms of the proteins of the invention which retain the ability to modulate apoptosis can be used. For example, a nucleic acid molecule of the present invention can comprise a nucleotide sequence which is at least about 75%, 80%, 85%, 90%, 95%, 96%, 97%, 98%, 99% or more identical to the nucleotide sequence to a nucleotide sequence encoding a protein of the invention (e.g., to the entire length of the nucleotide sequence encoding the protein), or a biologically active portion or complement of any of these nucleotide sequences.

Moreover, the nucleic acid molecule of the invention can comprise only a portion of the nucleic acid sequence encoding a polypeptide which modulates apoptosis. For example, a fragment of a nucleic acid sequence can be used as a probe or primer to measure transcription of a protein of the invention (e.g., the 130 kD leucine rich protein or a chaperone) as marker of apoptosis or a fragment encoding a portion of protein (e.g., a biologically active portion of a protein belonging to the Ian gene family) can be used as a modulating agent. A probe/primer of the invention typically comprises an internal fragment, e.g., a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence encoding the polypeptide.

Stimulatory agents which include only portions of the proteins of the invention retain biological activity and, therefore, comprise domains which mediate biological activity. For example, portions of Ian gene family members that retain the GTP binding domain and the carboxyterminal domain necessary for mitochondrial membrane localization can be used in the design of heterologous nucleic acid or protein sequences for use as stimulatory agents.

The invention further encompasses nucleic acid molecules that differ from the nucleotide sequences known in the art due to degeneracy of the genetic code and thus encode the same proteins as those encoded by the art-recognized sequence. In another embodiment, an isolated nucleic acid molecule of the invention has a nucleotide sequence encoding a protein having an amino acid sequence which differs by at least 1, but no greater than 5, 10, 20, 50 or 100 amino acid residues from an amino acid sequence encoding a polypeptide that modulates apoptosis as shown known in the art or as set forth herein.

Nucleic acid variants can be naturally occurring, such as allelic variants (same locus), homologues (different locus), and orthologues (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). Mutations can be introduced into a nucleotide sequence encoding a protein of the invention by standard techniques, such as site-directed mutagenesis or PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art.

Preferably, an isolated nucleic acid molecule for use as a modulator (or a probe/primer) hybridizes under stringent conditions to a nucleotide sequence encoding a protein of the invention. In one embodiment, such a sequence corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4, and 6. Additional stringent conditions can be found, e.g., in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4× sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991-1995), or alternatively 0.2×SSC, 1% SDS.

In a preferred embodiment, a variant protein of the invention can be assayed using techniques known in the art or described herein to verify that it retains its biologic activity, e.g., the ability to modulate apoptosis, the ability to modulate mitochondrial integrity, the ability to modulate mitochondrial inner membrane potential, or the ability to modulate expression or activity of other members of the apoptotic pathway.

In one embodiment, following isolation or amplification of a cDNA, the DNA fragment is introduced into an expression vector. As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid molecule in a form suitable for expression of the nucleic acid molecule in a host cell, which means that the recombinant expression vectors include one or more regulatory sequences, selected on the basis of the host cells to be used for expression and the level of expression desired, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel. 1990; Gene Expression Technology: Methods in Enzymology 185, Academic Press, San Diego, Calif. and Kaufman, R. 2000 Mol Biotechnol 16:151-60. Regulatory sequences include those which direct constitutive expression of a nucleotide sequence in many types of host cell, those which direct expression of the nucleotide sequence only in certain host cells (e.g., tissue-specific regulatory sequences) or those which direct expression of the nucleotide sequence only under certain conditions (e.g., inducible regulatory sequences).

It will be appreciated by those skilled in the art that the design of the expression vector may depend on such factors as the choice of the host cell to be transformed, the level of expression of protein desired, etc. When used in mammalian cells, the expression vector's control functions are often provided by viral regulatory elements. For example, commonly used promoters are derived from polyoma virus, adenovirus, cytomegalovirus and Simian Virus 40. Non-limiting examples of mammalian expression vectors include pCDM8 (Seed, B., (1987) Nature 329:840) and pMT2PC (Kaufman et al. (1987), EMBO J. 6:187-195). A variety of mammalian expression vectors carrying different regulatory sequences are commercially available. For constitutive expression of the nucleic acid in a mammalian host cell, a preferred regulatory element is the cytomegalovirus promoter/enhancer. Moreover, inducible regulatory systems for use in mammalian cells are known in the art, for example systems in which gene expression is regulated by heavy metal ions (see e.g., Mayo et al. (1982) Cell 29:99-108; Brinster et al. (1982) Nature 296:39-42; Searle et al. (1985) Mol. Cell. Biol. 5:1480-1489), heat shock (see e.g., Nouer et al. (1991) in Heat Shock Response, e.d. Nouer, L., CRC, Boca Raton, Fla., pp167-220), hormones (see e.g., Lee et al. (1981) Nature 294:228-232; Hynes et al. (1981) Proc. Natl. Acad. Sci. USA 78:2038-2042; Klock et al. (1987) Nature 329:734-736; Israel & Kaufman (1989) Nuc. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313). Still further, many tissue-specific regulatory sequences are known in the art, including the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916) and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Vector DNA can be introduced into mammalian cells via conventional transfection techniques. As used herein, the various forms of the term “transfection” are intended to refer to a variety of art-recognized techniques for introducing foreign nucleic acid (e.g., DNA) into mammalian host cells, including calcium phosphate co-precipitation, DEAE-dextran-mediated transfection, lipofection, or electroporation. Suitable methods for transfecting host cells can be found, e.g., in Sambrook et al. (Molecular Cloning: A Laboratory Manual, 2nd Edition, Cold Spring Harbor Laboratory press (1989)), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker may be introduced into a host cell on a separate vector from that encoding an Ian4 protein or, more preferably, on the same vector. Cells stably transfected with the introduced nucleic acid can be identified by drug selection (e.g., cells that have incorporated the selectable marker gene will survive, while the other cells die).

Another form of a stimulatory agent for stimulating expression or activity of a functional protein of the invention in a cell is a chemical compound that stimulates the expression or activity of an endogenous transcription factor that regulates expression of the functional gene in the cell. Such compounds can be identified using screening assays, e.g., that select for compounds that stimulate the expression of a protein of the invention or a reporter gene operably linked to the a promoter. Examples of suitable screening assays are described in further detail below.

B. Inhibitory Agents

According to the method of the invention, to inhibit expression and/or activity of one or more of the proteins of the invention, the cell is contacted with an inhibitory agent that inhibits expression and/or activity of one or more of the proteins.

In one embodiment, the expression and/or anti-apoptotic activity of a functional Ian gene or protein is inhibited in a cell that overexpresses it at inappropriate levels, in order to increase apoptosis.

In a more specific embodiment, agents that inhibit expression and/or activity of Ian4 can be used to enhance or increase apoptosis in a cell.

In another embodiment, the expression and/or activity of a non-functional Ian gene or protein (e.g. one containing a frameshift mutation) is inhibited in lymphoid cells, in order to decrease apoptosis.

In a more specific embodiment, the expression and/or activity of a non-functional Ian4 gene or protein (e.g. one containing a frameshift mutation) is inhibited in lymphoid cells, in order to decrease apoptosis.

In one embodiment, agents that inhibit expression and/or activity of one or more of leucine rich protein of 130 kD, hsp60, GRP78 or GRP94 can be used to reduce or inhibit apoptosis.

In a more specific embodiment, the expression and/or proapoptotic activity of functional leucine rich protein of 130 kD, GRP94, GRP78, or hsp60, is inhibited in a cell that overexpresses one or more of these molecules at inappropriate levels, in order to decrease apoptosis. In another embodiment, the expression and/or activity of a non-functional form of one of these molecules is inhibited in cells, in order to increase apoptosis.

In another embodiment, agents that inhibit expression, post-translational processing into the active form and/or activity of caspase 8 can be used to prevent receptor-independent apoptosis.

In a more specific embodiment, the pro-apoptotic activity of caspase 8 is inhibited in a cell that overexpresses the functional molecule at inappropriate levels, in order to decrease receptor-independent apoptosis. In another specific embodiment, the proapoptotic activity of non-functional caspase 8 is inhibited in cells, in order to increase receptor-independent apoptosis.

Inhibitory agents of the invention can be, for example, intracellular binding molecules that act to inhibit the expression or activity of a polypeptide. As used herein, the term “intracellular binding molecule” is intended to include molecules that act intracellularly to inhibit the expression or activity of a protein by binding to the protein or to a nucleic acid (e.g., an mRNA molecule) that encodes the protein. Examples of intracellular binding molecules, described in further detail below, include antisense nucleic acid molecules, siRNA, intracellular antibodies, and dominant negative inhibitors, e.g., deletion mutants.

i) Antisense Nucleic Acid Molecules:

In one embodiment, an inhibitory agent of the invention is an antisense nucleic acid molecule that is complementary to a gene encoding a protein of the invention. The use of antisense nucleic acid molecules to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA. An antisense nucleic acid for inhibiting in a cell the expression of a protein of the invention can be designed based upon the nucleotide sequence, as disclosed herein or known in the art, constructed according to the rules of Watson and Crick base pairing.

An antisense nucleic acid can exist in a variety of different forms. For example, the antisense nucleic acid can be an oligonucleotide that is complementary to only a portion of a gene. An antisense oligonucleotide can be constructed using chemical synthesis procedures known in the art. An antisense oligonucleotide can be chemically synthesized using naturally occurring nucleotides or variously modified nucleotides designed to increase the biological stability of the molecules or to increase the physical stability of the duplex formed between the antisense and sense nucleic acids, e.g. phosphorothioate derivatives and acridine substituted nucleotides can be used. To inhibit expression in cells in culture, one or more antisense oligonucleotides can be added to cells in culture media, typically at 200 μg oligonucleotide/ml.

Alternatively, an antisense nucleic acid molecule can be produced biologically using an expression vector into which a nucleic acid has been subcloned in an antisense orientation (i.e., nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

In another embodiment, an antisense nucleic acid for use as an inhibitory agent is a ribozyme. Ribozymes are catalytic RNA molecules with ribonuclease activity which are capable of cleaving a single-stranded nucleic acid, such as an mRNA, to which they have a complementary region (for reviews on ribozymes see e.g., Ohkawa, J. et al. (1995) J. Biochem. 118:251-258; Sigurdsson, S. T. and Eckstein, F. (1995) Trends Biotechnol. 13:286-289; Rossi, J. J. (1995) Trends Biotechnol. 13:301-306; Kiehntopf, M. et al. (1995) J. Mol. Med. 73:65-71). A ribozyme having specificity for mRNA encoding a polypeptide discussed herein can be designed based upon the nucleotide sequence encoding the polypeptide. For example, a derivative of a Tetrahymena L-19 IVS RNA can be constructed in which the base sequence of the active site is complementary to the base sequence to be cleaved in an mRNA. See for example U.S. Pat. Nos. 4,987,071 and 5,116,742, both by Cech et al. Alternatively, Ian4 mRNA can be used to select a catalytic RNA having a specific ribonuclease activity from a pool of RNA molecules. See for example Bartel, D. and Szostak, J. W. (1993) Science 261: 1411-1418.

ii) siRNA:

In another embodiment, an inhibitory agent of the invention is an siRNA molecule or other molecule that mediates RNAi (Scherr et al. 2003. Current Medicinal Chemistry 10:245). RNAi is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA.

For example, as demonstrated in the instant examples, an siRNA molecule comprising a sequence which corresponds to the sense strand of Ian4 can be used to inhibit Ian4 expression. In order to select sites in RNA molecules encoding proteins of the invention that are optimal for targeting, methods known in the art involving incubation of cellular extracts with oligonucleotides and cleavage by endogenous cellular Rnase H can be used (Scherr and Rossi. 1998. Nucleic Acids Res. 26:5079; Scherr et al. 2001. Mol. Ther. 4:454).

In one embodiment, the antisense RNA strand of RNAi can be antisense to at least a portion of the coding region of a protein of the invention or to at least a portion of the 5′ or 3′ untranslated region of the gene. In one embodiment, siRNA duplexes are composed of 21-nt sense and 21-nt antisense strands, paired in a manner to have a 2-nt 3′ overhang. In one embodiment, siRNA sequences with TT in the overhang. The target region can be, e.g., 50 to 100 nt downstream of the start codon, 3′-UTRs may also be targeted. In one embodiment, a 23-nt sequence motif AA(N 19)TT (N, any nucleotide) can be searched for and hits with between about 30-70% G/C-content can be selected. If no suitable sequences are found, the search is extended using the motif NA(N21). SiRNAs are preferably chemically synthesized using appropriately protected ribonucleoside phosphoramidites and a conventional DNA/RNA synthesizer. SiRNAs are also available commercially from, e.g., Dharmacon, Xeragon Inc, Proligo, and Ambion. In one embodiment one or more of the chemistries described above for use in antisense RNA can be employed.

In one embodiment, chemically or in vitro enzymatically synthesized siRNAs are delivered to target cells by transfection methods. In another embodiment, siRNAs can be expressed from expression cassettes using techniques that are known in the art.

iii) Antibodies:

Another type of inhibitory agent that can be used to inhibit the expression and/or activity of a protein in a cell is an antibody, e.g., an intracellular antibody, specific for a protein of the invention. The use of intracellular antibodies to inhibit protein function in a cell is known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Letters 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Bio/Technology 12:396-399; Chen, S-Y. et al. (1994) Human Gene Therapy 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al.).

To inhibit protein activity using an intracellular antibody, a recombinant expression vector is prepared which encodes the antibody chains in a form such that, upon introduction of the vector into a cell, the antibody chains are expressed as a functional antibody in an intracellular compartment of the cell.

To prepare an intracellular antibody expression vector, antibody light and heavy chain cDNAs encoding antibody chains specific for the target protein of interest, e.g., an Ian protein or other protein involved in apoptosis discussed herein, are isolated, typically from a hybridoma that secretes a monoclonal antibody specific for the protein. Antisera against a protein of the invention can be made as described herein or using techniques that are standard in the art. Antibodies can be prepared by immunizing a suitable subject, (e.g., rabbit, goat, mouse or other mammal) with a protein immunogen. An appropriate immunogenic preparation can contain, for examples, recombinantly expressed protein or a chemically synthesized peptide. The preparation can further include an adjuvant, such as Freund's complete or incomplete adjuvant, or similar immunostimulatory agent. Antibody-producing cells can be obtained from the subject and used to prepare monoclonal antibodies by standard techniques, such as the hybridoma technique originally described by Kohler and Milstein (1975, Nature 256:495-497) (see also, Brown et al. (1981) J. Immunol 127:539-46; Brown et al. (1980) J Biol Chem 255:4980-83; Yeh et al. (1976) PNAS 76:2927-31; and Yeh et al. (1982) Int. J. Cancer 29:269-75). The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med, 54:387-402; M. L. Gefter et al. (1977) Somatic Cell Genet., 3:231-36). Briefly, an immortal cell line (typically a myeloma) is fused to lymphocytes (typically splenocytes) from a mammal immunized with a protein immunogen as described above, and the culture supernatants of the resulting hybridoma cells are screened to identify a hybridoma producing a monoclonal antibody that binds specifically to the protein. Any of the many well known protocols used for fusing lymphocytes and immortalized cell lines can be applied for the purpose of generating a monoclonal antibody (see, e.g., G. Galfre et al. (1977) Nature 266:550-52; Gefter et al. Somatic Cell Genet., cited supra; Lerner, Yale J. Biol. Med., cited supra; Kenneth, Monoclonal Antibodies, cited supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody that specifically binds the protein of the invention are identified by screening the hybridoma culture supernatants for such antibodies, e.g., using a standard ELISA assay.

As an alternative to preparing monoclonal antibody-secreting hybridomas, a monoclonal antibody that binds to a protein of the invention can be identified and isolated by screening a recombinant combinatorial immunoglobulin library (e.g., an antibody phage display library) with the protein, or a peptide thereof, to thereby isolate immunoglobulin library members that bind specifically to the protein. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; Barbas et al. (1991) PNAS 88:7978-7982; and McCafferty et al. Nature (1990) 348:552-554.

Once a monoclonal antibody has been identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process. Nucleotide sequences of antibody light and heavy chain genes from which PCR primers or cDNA library probes can be prepared are known in the art. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database.

In another embodiment, fully human antibodies can be made using techniques that are known in the art. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make antibodies are described in U.S. Patents: U.S. Pat. Nos. 6,150,584; 6,458,592; 6,420,140. Other techniques are known in the art.

Once obtained, the antibody light and heavy chain sequences are cloned into a recombinant expression vector using standard methods. The expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In the most preferred embodiment, the vector encodes a single chain antibody (scFv) wherein the variable regions of the light and heavy chains are linked by a flexible peptide linker (e.g., (Gly₄Ser)₃) and expressed as a single chain molecule. To inhibit protein activity in a cell, the expression vector encoding the intracellular antibody is introduced into the cell by standard transfection methods, as discussed hereinbefore.

iv) Dominant Negative Inhibitors:

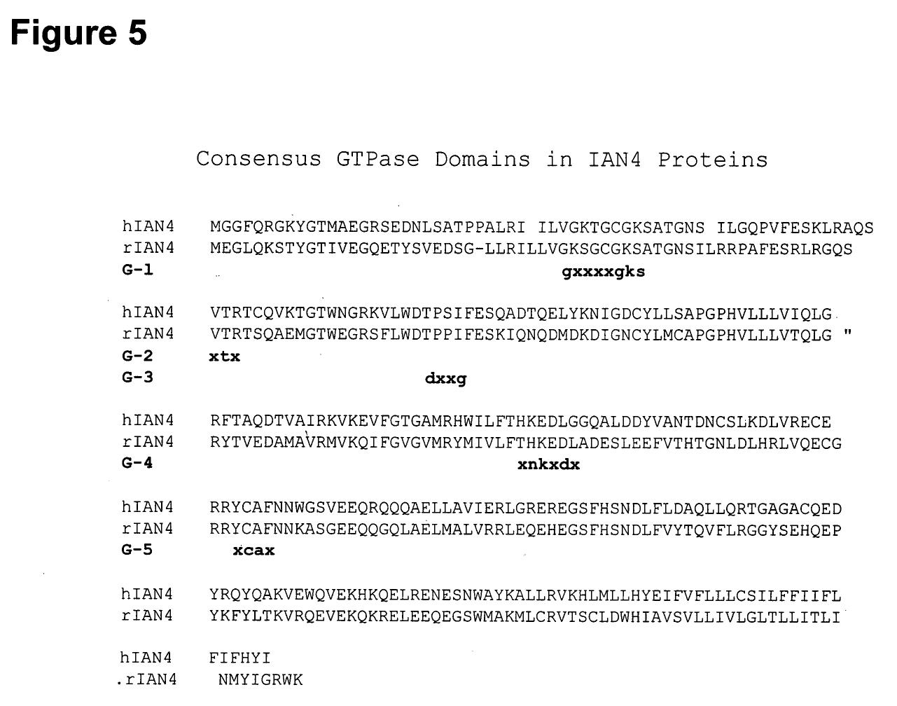

In another embodiment, an inhibitory agent of the invention can be a form of a protein that lacks the ability to modulate apoptosis. An exemplary dominant negative form of an a protein is an Ian 4 protein lacking at least the 20 carboxyterminal amino acids. The carboxyterminal 20 amino acids of Ian4 are necessary for localization of the protein in the outer membrane of mitochondria. Such dominant negative Ian4 proteins can be expressed in cells using a recombinant expression vector, which is introduced into the cell by standard transfection methods. FIG. 5 shows an alignment of rat Ian4 (GenBank AAL17698 gi:21591786) and human Ian4. In another embodiment, a dominant negative Ian4 protein can include a mutation in the sequence of a GTPase domain (e.g., one or more of the GTPase domains 1-5 shown in FIG. 5) that leads to reduced GTP binding and/or hydrolysis. For example, in one embodiment, a Ser in the G-1 GTPase domain of Ian4 is mutated to an Asn (Niemann et al. 2001 EMBO 20:5813).

In another embodiment, the activity of caspase 8 can be inhibited, using a known caspase 8 inhibitor, to modulate receptor-independent apoptosis.

v) Chemical Compounds:

Yet another type of inhibitory agent that can be used to inhibit the expression and/or activity of a protein of the invention in a cell is chemical compound that inhibits the expression or activity of an endogenous protein in the cell. Such compounds can be identified using screening assays that select for compounds such as small molecules or peptides that inhibit the expression or activity of a protein. Examples of suitable screening assays are described in further detail below.

The methods of the invention for modulating expression or activity of a protein of the invention in a cell can be practiced either in vitro or in vivo (the latter is discussed further in the following subsection). For practicing the method in vitro, cells can be obtained from a subject by standard methods and incubated (ie., cultured) in vitro with a stimulatory or inhibitory agent of the invention to inhibit or stimulate, respectively, apoptosis. For example, peripheral blood mononuclear cells (PBMCs) can be obtained from a subject and isolated by density gradient centrifugation, e.g., with Ficoll/Hypaque. Specific cell populations can be depleted or enriched using standard methods. For example, monocytes/macrophages can be isolated by adherence on plastic. T cells or B cells can be enriched or depleted, for example, by positive and/or negative selection using antibodies to T cell or B cell surface markers, for example by incubating cells with a specific primary monoclonal antibody (mAb), followed by isolation of cells that bind the mAb using magnetic beads coated with a secondary antibody that binds the primary mAb. Peripheral blood or bone marrow derived hematopoietic stem cells can be isolated by similar techniques using stem cell-specific mAbs (e.g., anti-CD34 mAbs). Specific cell populations can also be isolated by fluorescence activated cell sorting (FACS) according to standard methods. Monoclonal antibodies to cell-specific surface markers known in the art and many are commercially available. In an exemplary embodiment, T cells can be removed from a subject, treated with one or more stimulatory or inhibitory agents ex vivo to modulate apoptosis, and then returned to the subject.

III. Methods for Modulating Extpression or Activity of a Protein of the Invention in a Subject

Another aspect of the invention pertains to in vivo methods for modulating apoptosis in a subject (e.g., in vivo methods). The term “subject” is intended to include living organisms in which an immune response can be elicited. Preferred subjects are mammals. Examples of subjects include humans, monkeys, dogs, cats, mice, rats, cows, horses, goats and sheep. As discussed above, one way to modulate apoptosis in a subject is to treat cells (e.g., T cells) ex vivo with one or more modulatory agents of the invention, such that apoptosis of the cells is modulated, followed by administration of the cells to the subject. In another embodiment, apoptosis is modulated in a subject by administering to the subject one or more of the modulatory agents of the invention.

For stimulatory or inhibitory agents that comprise nucleic acid molecules (including recombinant expression vectors, antisense RNA, siRNA, intracellular antibodies or dominant negative inhibitors), the agents can be introduced into cells of the subject using methods known in the art for introducing nucleic acid (e.g., DNA) into cells in vivo. Examples of such methods include:

Direct Injection: Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo can be used. Such an apparatus is commercially available (e.g., from BioRad).

Receptor-Mediated DNA Uptake: Naked DNA can also be introduced into cells in vivo by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Retroviruses: Defective retroviruses are well characterized for use in gene transfer for gene therapy purposes (for a review see Miller, A. D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay.

A modulatory agent, such as a chemical compound that stimulates or inhibits endogenous polypeptide activity, can be administered to a subject as a pharmaceutical composition. Such compositions typically comprise the modulatory agent and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifingal agents, for example, parabens, chlorobutanol, phenyl, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In one embodiment, the modulatory compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of individuals.

IV. Applications of the Methods of the Invention

Identification of Ian4 as an anti-apoptotic factor that functions exclusively in mature T cells allows for manipulation of cell survival in a variety of clinical situations using the modulatory methods of the invention. In addition, the invention also identifies hsp60, leucine rich protein of 130 kD, GRP78 and GRP94 as molecules which modulate apoptosis.

Accordingly, the invention provides for diagnostic methods in which expression and/or activity of one or more of the proteins of the invention is used as a marker of apoptosis. Expression of these molecules can be detected, e.g., at the nucleic acid level or at the protein level using techniques known in the art (e.g., northern blots, western blots, etc.)

The methods of the invention that involve stimulation of the expression and/or anti-apoptotic activity functional Ian gene family members result in reduced apoptosis or enhanced cell survival. In addition, as set forth above, methods of the invention that involve inhibition of non-functional Ian gene family members, also result in reduced apoptosis. Methods of the invention that involve modulation of hsp60, leucine rich protein of 130 kD, GRP78, or GRP94 result in modulation of apoptosis. Further, the methods of the invention that involve stimulation of caspase 8 expression, post-translational modification, or activity result in increased receptor-independent apoptosis.

In contrast, the methods of the invention that inhibit the expression and/or anti-apoptotic activity of functional Ian gene family members result in enhanced apoptosis. Agents that reduce or inhibit the expression, post-translational processing, or activity of caspase 8 lead to a decrease in receptor-independent apoptosis. Thus, to treat a disease condition where reduced apoptosis is beneficial (e.g., in lymphopenia, and other immune disorders), functional Ian expression or activity can be upmodulated or hsp60, leucine rich protein of 130 kD, GRP78, or GRP94 expression or activity can be downmodulated to thereby decrease apoptosis. Caspase 8 expression, post-translational processing, or activity can be reduced to decrease or inhibit receptor-independent apoptosis. Alternatively, to treat a disease condition where enhanced apoptosis is beneficial (e.g., in lymphoid neoplasms), functional Ian4 expression or activity can be downmodulated or hsp60, leucine rich protein of 130 kD, GRP78, or GRP94 expression or activity can be modulated (e.g., upregulated) to thereby increase apoptosis. Caspase 8 expression, post-translational processing, or activity can be increased to increase receptor-independent apoptosis.

Application of the methods of the invention to the treatment of disease conditions may result in cure of the condition, a decrease in the type or number of symptoms associated with the condition, either in the long term or short term (i.e., amelioration of the condition) or simply a transient beneficial effect to the subject.

Numerous disease conditions associated with reduced cell survival or enhanced cell survival are known in to those of skill in the art and could benefit from modulation of the type of response mounted in the individual suffering from the disease condition. Application of the immunomodulatory methods of the invention to such disorders/diseases is described in further detail below.

A. Autoimmune Diseases

In one embodiment, Ian expression or activity can be downmodulated or hsp60, leucine rich protein of 130 kD, GRP78, or GRP94 expression or activity can be upmodulated to increase apoptosis. In another embodiment, Caspase 8 expression, post-translational processing, or activity can be increased to increase receptor-independent apoptosis. This can be of use therapeutically in the treatment of autoimmune diseases. Many autoimmune disorders are the result of inappropriate activation of T cells that are reactive against self tissue and that promote the production of cytokines and autoantibodies involved in the pathology of the diseases.

Accordingly, increased apoptosis of cells involved in such undesirable immune responses would be of benefit, for example, methods of enhancing apoptosis in subjects suffering from, or susceptible to, an autoimmune disease could be performed in such a subject. The method can involve either direct administration of a modulatory agent to the subject or ex vivo treatment of cells obtained from the subject (e.g., Th2, Th1 cells, B cells, non-lymphoid cells) with a modulatory agent followed by readministration of the cells to the subject. The treatment may be further enhanced by administering other agents to enhance apoptosis or decrease proliferation to the subject.

Non-limiting examples of autoimmune diseases and disorders having an autoimmune component that may be treated according to the invention include arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

In another embodiment, decreased apoptosis can be of benefit in treating certain autoimmune disorders or diseases. For example, as demonstrated herein, loss of Ian4 activity can lead to the development of type I diabetes and/or lymphopenia. Accordingly, in one embodiment, type I diabetes and/or lymphopenia can be treated by increasing Ian expression or activity and modulating (e.g., downregulating) expression or activity (e.g., por-activity) of leucine rich protein of 130 kD, hsp60, GRP78, or GRP94 to decrease apoptosis in mature T cells. This may be done by administrating an agent that decreases apoptosis to a subject.

The efficacy of agents for treating autoimmune diseases can be tested animal models of human diseases (e.g., EAE as a model of multiple sclerosis and the NOD mice as a model for diabetes) or other well characterized animal models of human autoimmune diseases. Such animal models include the mrl/lpr/lpr mouse as a model for lupus erythematosus, murine collagen-induced arthritis as a model for rheumatoid arthritis, and murine experimental myasthenia gravis (see Paul ed., Fundamental Immunology, Raven Press, New York, 1989, pp. 840-856), and the BB rat as a model of type I diabetes. A modulatory (i.e., stimulatory or inhibitory) agent of the invention is administered to test animals and the course of the disease in the test animals is then monitored by the standard methods for the particular model being used. Effectiveness of the modulatory agent is evidenced by amelioration of the disease condition in animals treated with the agent as compared to untreated animals (or animals treated with a control agent).

B. Cancer

Increasing apoptosis of neoplastic cells, e.g., tumor cells or cancer cells, is one method of controlling unwanted proliferation. In one embodiment, the subject methods are used to treat leukemia (e.g., chronic or acute lymphoblastic leukemia) or lymphoma (e.g., B-cell lymphoma, T-cell lymphoma, Hodgkins lymphoma, non-Hodgkins lymphoma, hairy cell lymphoma, or Burkett's lymphoma). Methods of inhibiting Ian expression and/or activity or methods of modulating(e.g., upregulating) leucine rich protein of 130 kD, hsp60, GRP78, or GRP94 expression and/or activity can be used to increase apoptosis and ameliorate the course of the disease. These methods can involve either direct administration of an agent to a subject with cancer or ex vivo treatment of cells obtained from the subject with an agent followed by readministration of the cells to the subject. The treatment may be further enhanced by further administering other agents known in the art to enhance treatment of cancer (e.g., chemotherapeutic agents or antibody-mediated therapies). In one embodiment, methods of the invention involving administration of the subject modulatory agents allow for administration of chemotherapeutic agents at reduced concentration, thereby reducing their toxicity.

C. Immunosuppressed or Immunocompromised States

In another embodiment, the methods of the invention can be used to decrease apoptosis in lymphocytic cells. Apoptosis of immune cells can lead to certain immunosupressed or immunocomprised states, e.g., lymphopenia. Accordingly, in one embodiment, methods of stimulating Ian4 expression and/or activity or methods of modulating leucine rich protein of 130 kD, hsp60, GRP78, or GRP94 expression and/or activity can be used to decrease apoptosis and ameliorate the course of the disease. These methods can involve either direct administration of an agent to a subject or ex vivo treatment of cells obtained from the subject with an agent followed by readministration of the cells to the subject. In one embodiment, the subject methods can be used to treat lymphopenia associated with AIDS or other viral infections. In another embodiment, the subject methods can be used to treat subjects that are immunosuppressed following treatment with a drug or with radiation (e.g., external beam radiation or internal radiation from radiolabeled antibodies). The treatment may be further enhanced by administering other agents known in the art to enhance survival of lymphocytes (e.g., cytokine based therapies).

In addition to the foregoing disease situations, the modulatory methods of the invention also are useful for other purposes. For example, the modulatory methods of the invention can be applied to vaccinations. That is, the agents of the invention that decrease or inhibit apoptosis can serve as adjuvants in an immune response to an antigen.

V. Compositions for Modulating Apoptosis

Another aspect of the invention pertains to compositions that can be used to modulate Ian proteins (e.g., Ian4) or hsp60, leucine rich protein of 130 kD, GRP78, GRP94, and/or caspase 8 in accordance with the methods of the invention. The invention provides recombinant expression vectors comprising a nucleotide sequence encoding a protein operatively linked to regulatory sequences that direct expression of the protein specifically in certain cell types. In one embodiment, the regulatory sequences direct expression of the Ian4 protein specifically in lymphoid cells (e.g., T cells or B cells). In one embodiment, the lymphoid cells are T cells. T cell specific regulatory elements are known in the art, such as the promoter regulatory region of T cell receptor genes (see e.g., Winoto and Baltimore (1989) EMBO J. 8:729-733; Leiden, J. M. (1994) Annu. Rev. Immunol. 11:539-570; Hettman, T. and Cohen, A. (1994) Mol. Immunol. 31:315-322; Redondo, J. M. et al. (1991) Mol. Cell. Biol. 11:5671-5680). Other examples of T cell specific regulatory elements are those derived from the CD3 gene (see e.g., Clevers, H. et al. (1988) Proc. Natl. Acad. Sci. USA 85:8623-8627; Clevers, H. C. et al. (1988) Proc. Natl. Acad. Sci. USA 85:8156-8160; Georgopoulos, K. et al. (1988) EMBO J. 7:2401-2407), the CD4 gene (see e.g., Sawada, S. and Littman, D. R. (1991) Mol. Cell. Biol. 11:5506-5515; Salmon, P. et al. (1993) Proc. Natl. Acad. Sci. USA 90:7739-7743; Hanna, Z. et al. (1994) Mol. Cell. Biol. 14:1084-1094; see also GenBank accession numbers U01066 and S68043 for human CD4 promoter sequences) and the CD2 gene (see e.g., Zhumabekov, T. et al. (1995) J. Immunol. Methods 185:133-140). A DNA fragment comprising one or more T cell specific regulatory elements, such as a promoter and enhancer of a T cell receptor gene, can be obtained by standard molecular biology methods, such as by PCR using oligonucleotide primers corresponding to the 5′ and 3′ ends of the desired region and genomic DNA from T cells as the template. Once the DNA fragment comprising T cell specific regulatory elements is obtained, it can be operatively linked to a cDNA encoding a protein (e.g., the two DNA fragments can be ligated together such that the regulatory elements are located 5′ of the sequences) and introduced into vector, such as a plasmid vector, using standard molecular biology techniques.

In another embodiment, the lymphoid cells are B cells (i.e., within the recombinant expression vector the nucleotide sequences encoding a protein are operatively linked to regulatory sequences that direct expression of the protein specifically in B cells). B cell specific regulatory elements are known in the art, such as the promoter regulatory region of immunoglobulin genes (see e.g., Banerji et al. (1983) Cell 33:729-740; Queen and Baltimore (1983) Cell 33:741-748). Other examples of B cell specific regulatory elements are those derived from the CD20 (BI) gene (see e.g., Thevenin, C. et al. (1993) J. Biol. Chem. 268:5949-5956; Rieckmann, P. et al. (1991) J. Immunol. 147:3994-3999), the Fc epsilon RIIa gene (see e.g., Suter, U. et al. (1989) J. Immunol. 143:3087-3092) and major histocompatibility class II genes (see e.g., Glimcher, L. H. and Kara, C. J. (1992) Annu. Rev. Immunol. 10:13-49; Benoist, C. and Mathis, D. (1990) Annu. Rev. Immunol. 8:681-715). A DNA fragment comprising B cell specific regulatory elements, such as a promoter and enhancer of an immunoglobulin gene, can be obtained by standard molecular biology methods, such as by PCR using oligonucleotide primers corresponding to the 5′ and 3′ ends of the desired region and genomic DNA from B cells as the template. Once the DNA fragment comprising B cell specific regulatory elements is obtained, it can be operatively linked to a cDNA encoding a protein (e.g., the two DNA fragments can be ligated together such that the regulatory elements are located 5′ of the Ian4 sequences) and introduced into vector, such as a plasmid vector, using standard molecular biology techniques.

In yet another embodiment, the invention provides recombinant expression vectors comprising a nucleotide sequence encoding a protein operatively linked to regulatory sequences that direct expression of the protein specifically in hematopoietic stem cells. Hematopoietic stem cell specific regulatory elements are known in the art. Preferably regulatory elements derived from the CD34 gene are used (see e.g., Satterthwaite, A. B. et al. (1992) Genomics 12:788-794; Burn, T. C. et al. (1992) Blood 80:3051-3059).

Another aspect of the invention pertains to recombinant host cells that express a protein of the invention. The terms “host cell” and “recombinant host cell” are used interchangeably herein to refer to a cell into which a recombinant expression vector has been introduced. It is understood that such terms refer not only to the particular subject cell but to the progeny of such a cell. Because certain modifications may occur in succeeding generations due to either mutation or environmental influences, such progeny may not, in fact, be identical to the parent cell, but as long as these progeny cells retain the recombinant expression vector, these progeny are still intended to be included within the scope of the term “host cell” as used herein.

In one embodiment, the invention provides a host cell into which a recombinant expression vector encoding a protein of the invention has been introduced. The host cell can be, e.g., a T cell or a B cell. Exemplary host T cells of the invention include, for example a T cell clone that is cultured in vitro (such as those described in the Examples) or, alternatively, a normal T cell that is isolated from a subject (e.g., a peripheral blood T cell or a splenic T cell). Standard methods for preparing and culturing T cell clones in vitro, or isolating T cells (e.g., from peripheral blood) are known in the art, for example through the use of mAbs that bind T cell specific cell surface markers (e.g., CD3) or surface markers for specific subsets of T cells (e.g., CD4 or CD8). The recombinant expression vector can be introduced into the T cell by one of a variety of known transfection methods for introducing DNA into mammalian cells, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual, 2nd Edition, Cold Spring Harbor Laboratory press (1989)), and other laboratory manuals.

In another embodiment, the host lymphoid cell of the invention is a host B cell into which a recombinant expression vector encoding a protein of the invention has been introduced. The B cell can be, for example a B lymphoma cell that is cultured in vitro (such as M12 cells as described in the Examples) or, alternatively, a normal B cell that is isolated from a subject (e.g., a peripheral blood B cell or a splenic B cell). Various B lymphoma cell lines are available in the art and standard methods for culturing such cells in vitro are known. Additionally, standard methods for isolating normal B cells (e.g., from peripheral blood) are known in the art, for example through the use of mAbs that bind B cell specific cell surface markers (e.g., membrane immunoglobulin, B7-1, CD20). The recombinant expression vector can be introduced into the B cell by standard methods, as described above for T cells.

In yet another embodiment, the invention provides a host hematopoietic stem cell into which a recombinant expression vector encoding a protein of the invention has been introduced. Hematopoietic stem cells can be isolated from a subject (e.g., from peripheral blood or bone marrow of the subject) using standard methods known in the art for isolating such stem cells, for example through the use of mAbs that bind hematopoietic stem cell specific cell surface markers, preferably CD34 (for further descriptions of isolation of stem cells, see e.g., Wagner, J. E. et al. (1995) Blood 86:512-523; Murray, L. et al. (1995) Blood 85:368-378; Bernardi, A. C. et al. (1995) Science 267:104-108; Bernstein, I. D. et al. (1994) Blood Cells 20:15-24; Angelini, A. et al. (1993) Int. J. Artif Organs 16 Suppl. 5:13-18; Kato, K. and Radburch, A. (1993) Cytometry 14:384-392; Lebkowski, J. S. et al. (1992) Transplantation 53:1011-1019; Lebkowski, J. et al. (1993) J. Hematother. 2:339-342). The recombinant expression vector can be introduced into the hematopoietic stem cell by standard methods, as described above for T cells.

Compositions comprising combinations of modulatory agents are also provided by the invention.

Kits for modulating apoptosis are also encompassed by the invention. In one embodiment, a kit of the invention comprises at least one modulatory agent of the invention packaged with instructions for using the modulatory agent to modulate apoptosis. In another embodiment, the kit comprises at least one modulatory agent for use in modulating apoptosis and a second agent known in the art to modulate apoptosis. Combination kits, comprising two or more of the modulatory (e.g., stimulatory or inhibitory) agents of the invention are also provided.

VI. Screening Assays

Another aspect of the invention pertains to screening assays for identifying compounds or agents that modulate the expression and/or activity of the proteins of the invention, e.g., Ian4, hsp60, leucine rich protein of 130 kD, GRP78, GRP94, and caspase 8. In various embodiments, these screening assays can identify, for example, compounds that modulate the expression or functional activity of the proteins, proteins that interact with the proteins of the invention, as well as compounds that modulate these protein-protein interactions.

Any number of test compounds, e.g., peptidomimetics, small molecules or other drugs can be used for testing and can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In many drug screening programs which test libraries of modulating agents and natural extracts, high throughput assays are desirable in order to maximize the number of modulating agents surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test modulating agent. Moreover, the effects of cellular toxicity and/or bioavailability of the test modulating agent can be generally ignored in the in vitro system, the assay instead being focused primarily on the effect of the drug on the molecular target as may be manifest in an alteration of binding affinity with upstream or downstream elements.

In one embodiment, the invention provides assays for screening candidate or test compounds which bind to or modulate the activity of a polypeptide of the invention or biologically active portion thereof.

Assays can be used to screen for modulating agents, including homologs, which are either agonists or antagonists of the normal cellular function of the subject polypeptides. For example, the invention provides a method in which an indicator composition is provided which comprises a protein which modulates apoptosis, i.e., Ian4, hsp60, leucine rich protein of 130 kD, GRP78 GRP94, or caspase 8. The indicator composition can be contacted with a test compound. The effect of the test compound on activity, as measured by a change in the indicator composition, can then be determined to thereby identify a compound that modulates the activity of a protein of the invention. A statistically significant change, such as a decrease or increase, in the level of protein expression or activity in the presence of the test compound (relative to what is detected in the absence of the test compound) is indicative of the test compound being a modulating agent. The indicator composition can be, for example, a cell or a cell extract.

i) Cell-Based Assays

Cell-based assays for identifying small molecule agonists/antagonists and the like can be used to identify additional modulating agents for use in the claimed invention. For example, cells expressing a protein of the invention can be used in a screening assay. In a preferred embodiment, cells can be caused to express or overexpress a recombinant protein in the presence and absence of a test agent of interest, with the assay scoring for modulation in the expression, post-translational modification, or activity of the protein of the invention in the target cell mediated by the test agent. For example, modulating agents which produce a statistically significant change in expression or activity of a protein of the invention (either an increase or decrease) can be identified.

Recombinant expression vectors that can be used for expression of proteins of the invention are known in the art (see discussions above). In one embodiment, within the expression vector the protein coding sequences are operatively linked to regulatory sequences that allow for constitutive or inducible expression of the protein in the indicator cell(s). Use of a recombinant expression vector that allows for constitutive or inducible expression of a protein in a cell is preferred for identification of compounds that enhance or inhibit the activity of a protein of the invention. In an alternative embodiment, within the expression vector the protein coding sequences are operatively linked to regulatory sequences of the endogenous gene (i.e., the promoter regulatory region derived from the endogenous gene). Use of a recombinant expression vector in which protein expression is controlled by the endogenous regulatory sequences is preferred for identification of compounds that enhance or inhibit the transcriptional expression of a protein of the invention.

In one embodiment, an assay is a cell-based assay comprising contacting a cell expressing a protein of the invention with a test compound and determining the ability of the test compound to modulate (e.g. stimulate or inhibit) the activity of the protein molecule. Determining the ability of the test compound to modulate the activity of the protein can be accomplished using a variety of readouts.

Direct Readouts:

In one embodiment, the direct effect of a test compound on apoptosis is measured. For example, determining the ability of the test agent to modulate the activity of a polypeptide of the invention can be accomplished, for example, by measuring the ability of a test compound to modulate apoptosis in a cell, e.g., in a T cell. The hallmark of apoptosis is degradation of DNA. Early in the process, this degradation occurs in internucleosomal DNA linker regions. The DNA cleavage may yield double-stranded and single-stranded DNA breaks. Apoptosis can be measured in cells using standard techniques. Therefore, degradation of genomic DNA of a population of cells can be analyzed by agarose gel electrophoresis, or DNA fragmentation assays based on 3H-thymidine or 5-Bromo-2′-deoxy-uridine can be used.

To analyze apoptosis in individual cells, apoptotic cells may be recognized microscopically because of the characteristic appearance of nuclear chromatin condensation and fragmentation. Apoptosis can be measured in individual cells, for example, using Hoechst stain and looking for cells with pyknotic nuclei as described in the appended Examples. Alternatively, double and single-stranded DNA breaks can be detected by labeling the free 3′-OH termini with modified nucleotides (e.g., biotin-dUTP, DIG-dUTP, fluorescein-dUTP) in an enzymatic reaction. Terminal deoxynucleotidyl transferase (TdT) catalyzes the template independent polymerization of deoxyribonucleotides to the 3′ end of the DNA. This method is referred to as TUNEL (TdT-mediated dUTP-X nick end labeling). Alternatively, free 3′ OH groups may be labeled using DNA polymerases by nick translation. Tunnel staining can be used to identify cells with double stranded DNA breaks. Labeled free 3′ OH groups that have incorporated labeled dUTP can be visualized by flow cytometry and/or fluorescence microscopy. Reagents for performing these assays are available e.g., from Roche Molecular Biochemicals USA (In situ cell death detection kit). In addition, annexin (e.g., Annexin-V-Alexa™ 568 commercially available from Roch molecular Biochemicals USA) can be used for this purpose. One of the early plasma membrane changes associated with cells undergoing apoptosis is the translocation of phosphatidylserine from the inner leaflet of the plasma membrane to the outer layer, thereby exposing phosphatidylserine at the surface of the cell. Annexin-V is a phospholipid binding protein which binds to phosphatidyl serine and can be used as a probe for phosphatidylserine on cell surfaces. Annexin-V can be used in combination with a DNA stain (e.g., BOBO™-1) to differentiate apoptotic cells from necrotic cells.

Indirect Assays:

Assays can also be devised to indirectly assess the effects of a modulating agent on apoptosis by measuring the effects of the modulating agent on the expression and/or intracellular localization of components of the apoptotic pathway. In one embodiment, the DNA or protein encoding an apoptotic pathway component of the agent or a fragment thereof can be functionally linked or fused to a reporter gene. When expressed in a suitable expression vector, the intracellular distribution of the resultant fusion protein can then be localized by assaying its expression or within the cell using techniques well known in the art. Exemplary “reporter genes” include Beta-galactosidase (β-gal), Beta-glucoronidase (GUS), firefly luciferase (ex. those from P. pyralis), or Green Fluorescent Proteins (GFP) from the bioluminescent jellyfish A. victoria. In one embodiment, the reporter gene can be fused to an an apoptogenic factors (e.g. procaspase-2, procaspase-3, procaspase-8, procaspase-9, cytochrome-c, Smac/Diablo, AIF, or endonuclease G) to measure release of the protein from mitochondria. Since apoptogenic factors are normally confined to the intermembrane space or matrix of mitochondria, the release of these factors from mitochondria can be used to assess the effects of a modulating agent on mitochondrial integrity. Methods for assessing the mitochondrial release of cytochrome are disclosed in the art (see Heiskanen et al, 1999, J. Biol. Chem, 274: 5654).

In another embodiment, the effects of modulating agents on apoptosis can be assessed indirectly by their effects on the expression and/or activity of proteins of the invention. In one preferred embodiment, the effects of modulating agents on Ian activity can be assessed by measuring mitochondrial membrane permeabilization. For example, mitochondrial inner membrane permeabilization can be measured in intact cells by loading the cytosol or the mitochondrial matrix with a die that does not normally cross the inner membrane, e.g., calcein (Bernardi et al. 1999. Eur. J. Biochem. 264:687; Lemasters, J., J. et al. 1998. Biochem. Biophys. Acta 1366:177. In another embodiment, mitochondrial inner membrane permeabilization can be assessed by determining a change in the mitochondrial inner membrane potential (ΔΨm) with potentiometric indicator dyes. For example, cells can be incubated with lipophilic cationic fluorochromes such as DiOC6 (Gross et al. 1999. Genes Dev. 13:1988) (3,3′dihexyloxacarbocyanine iodide) or JC-1(5,5′,6,6′-tetrachloro-1,1′, 3,3′-tetraethylbenzimidazolylcarbocyanine iodide). These dyes accumulate in the mitochondrial matrix, driven by the Ψm. Dissipation results in a reduction of the fluorescence intensity (e.g., for DiOC6 (Gross et al. 1999. Genes Dev. 13:1988) or a shift in the emission spectrum of the dye. These changes can be measured by cytofluorometry or microscopy of whole cells or purified mitochondria. In a preferred embodiment, a dye such as Mitolight™ or MitoTracker™ (Molecular Probes, Eugene, Oreg.) can be used as described in the instant examples. As list of alternative potentiometic dyes which may be used to assess inner mitochondrial membrane permeabilization include those listed in Table I:

TABLE I
Potentiometric Indicators

| | Structure | |
|---|---|---|
| Dyes | (Charge) | Optical Response |
| ANEP dyes | Styryl | FAST; fluorescence |
| (aminonaphthyl- | (cationic or | excitation ratio 440/505 nm |
| ethenylpyridinium | zwitterionic) | decreases upon membrane |
| | | depolarization |
| RH dyes | Styryl | FAST; generally similar to |
| (Rina Hildesheim) | (cationic or | ANEP dyes with excitation |
| | zwitterionic) | red shift upon membrane |
| | | hyperpolarization |
| Impermeant Oxonols | Hybrid oxonol | FAST; absorbance changes |
| (oxonol dyes with | (anionic) | upon membrane |
| phenylsulfonate | | hyperpolarization |
| substituents | | |
| Carbocyanines | Carbocyanine | SLOW; fluorescence generlly |
| (Indo- DiI), thia | (cationic) | decreases upon membrane |
| (DiS) and oxa- (DiO) | | hyperpolarization |
| carbocyanines with | | |
| short (n = 1 − 7) | | |
| alkyl tails | | |
| Rhodamine and the | Rhodamine | SLOW; used to obtain |
| methyl and ethyl | (cationic) | unbiased images of potential- |
| esters of | | |
| tetramethylrfodamine | | dependent dye distribution |
| Oxonols | Oxonol | SLOW; flourescence |
| | (anionic) | decreases upon membrane |
| | | hyperpolarization |
| DiBAC Dyes | Oxonol | SLOW; flourescence |
| (bis-barbituric | (anionic) | decreases upon membrane |
| acid oxonols) | | hyperpolarization |

It is anticipated that fluorescent and/or chromogenic pH indicators can also be utilized to assess the integrity of the inner mitochondrial membrane. As used herein, the term “chromogenic indicator” refers to a compound which produces a colorometric readout as an indicator of pH. Examples of chromogenic indicators which change their color in response to a change in pH include, but are not limited to, litmus, phenolphthalein, and phenyl red. In another embodiment, the Mg²⁺ dye, Magfure, is used to measure citrate entry via CIC due to the ability of citrate to chelate Mg²⁺.

Also, according to the present invention, a fluorescent indicator can be utilized in accordance with the present invention. As used herein, the term “fluorescent indicator” refers to a compound which produces a fluorescent readout as an indicator of pH. In a preferred embodiment, the change in the fluorescent readout of such an indicator is a quantitative measurement of the change in, for example, pH or membrane potential. Examples of fluorescent indicators which produce a change in fluorescence in response to a change in pH include, but are not limited to, fluorescein and fluorescein derivatives (e.g., fluorescein diacetate (FDA), carboxyfluorescein diacetate (CFDA), the polar fluorescein derivative, BCECF, and the AM ester of BCECF, methylated fluorescein and/or fluorescein diacetate, and fluorescein sulfonic acid and/or fluorescein sulfonic acid diacetate); the seminaphthorhodafluors (SNARF dyes) and seminapthofluoresceins (SNAFL dyes); and/or pyranin. The typical pH ranges and spectral measurements for exemplary pH indicators are provided in Table 2.

TABLE 2
pH Indicators

| Parent Flurophore | pH Range | Typical Measurement |
|---|---|---|
| SNAFL indicators | 7.2-8.2 | Excitation Ratio 490/540 nm |
| | | or emission ratio 540/630 nm |
| SNARF indicators | 7.0-8.0 | Emmission ratio 580/640 nm |
| HPTS (pyranine) | 7.0-8.0 | Excitation ratio 450/405 nm |
| BCECF | 6.5-7.5 | Excitation ratio 490/450 nm |
| Fluoresceins and | 6.0-7.2 | Excitation ratio 490/450 nm |
| carboxyfluorosceins | | |

In another embodiment, modulators of protein expression are identified in a method wherein a cell is contacted with a candidate compound and the expression of mRNA or protein in the cell is determined. The level of expression of mRNA or protein in the presence of the candidate compound is compared to the level of expression of mRNA or protein in the absence of the candidate compound. The candidate compound can then be identified as a modulator of the expression of a protein of the invention based on this comparison. For example, when expression of mRNA or protein is greater (e.g., statistically significantly greater) in the presence of the candidate compound than in its absence, the candidate compound is identified as a stimulator of mRNA or protein expression. Alternatively, when expression of mRNA or protein is less (e.g., statistically significantly less) in the presence of the candidate compound than in its absence, the candidate compound is identified as an inhibitor of mRNA or protein expression. The level of mRNA or protein expression in the cells can be determined by methods known in the art for detecting mRNA or protein.

In one embodiment, cells are not subjected to a stimulus that induces apoptosis prior to contacting them with a test compound. In one embodiment, cells are subjected to a stimulus that induces apoptosis prior to contacting them with a test compound. Exemplary stimuli include those that cause receptor-mediated apoptosis (e.g., ligation of CD95, DR3/WSL, or (TRAIL)/APO-2L receptors) or those that cause receptor-independent apoptosis (e.g., withdrawal of a growth factor or cytokine).

In one embodiment, a cell for use in a screening assay expresses a non-functional form of Ian4, for example, a form of Ian4 comprising a carboxyterminal deletion.

In yet another aspect of the invention proteins of the invention or portions thereof can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with proteins of the invention and are involved in their activity.

The two-hybrid system is based on the modular nature of most transcription factors, which consist of separable DNA-binding and activation domains. Briefly, the assay utilizes two different DNA constructs. In one construct, the gene that codes for an Ian4 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with a protein of the invention. Once interacting proteins have been identified, the ability of a test compound to modulate the interaction between a protein of the invention and the interacting protein can be tested using techniques that are known in the art, e.g., using the cell-free assays described herein.

ii) Cell-Free Assays

In one embodiment, the assay is a cell-free assay in which a protein of the invention or biologically active portion thereof is contacted with a test compound and the ability of the test compound to modulate (e.g., stimulate or inhibit) the activity of the protein or biologically active portion thereof is determined. The protein can be provided as a lysate of cells that express the protein, as a purified or semipurified polypeptide, or as a recombinantly expressed polypeptide. In one embodiment, a cell-free assay system further comprises a cell extract or isolated components of a cell, such as mitochondria. Such cellular components can be isolated using techniques which are known in the art. Determining the ability of the test compound to modulate the activity of a protein can be accomplished, for example, by determining the ability of the protein to bind to a target molecule (another molecule to which the protein of the invention binds) using an art recognized method for determining direct binding. Determining the ability of the protein to bind to a target molecule can be accomplished, e.g., using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In yet another embodiment, the cell-free assay involves contacting a protein or biologically active portion thereof with an interacting molecule which binds the protein to form an assay mixture, contacting the assay mixture with a test compound, and determining the ability of the test compound to interact with one of the components of the mixture or to disrupt formation of a complex between the two components, wherein determining the ability of the test compound to interact with the protein comprises the ability of the compound to interact with one of the components or disrupt formation of a complex indicates that the compound interferes with a binding activity of the protein.

The cell-free assays of the present invention are amenable to use of both soluble and/or membrane-bound forms of proteins. In the case of cell-free assays in which a membrane-bound form a protein is used it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Suitable assays are known in the art that allow for the detection of protein-protein interactions (e.g., immunoprecipitations, two-hybrid assays and the like). By performing such assays in the presence and absence of test compounds, these assays can be used to identify compounds that modulate (e.g., inhibit or enhance) the interaction of a protein of the invention with a target molecule(s).

Determining the ability of the protein to bind to or interact with a target molecule can be accomplished, e.g., by direct binding. In a direct binding assay, the protein could be coupled with a radioisotope or enzymatic label such that binding of the protein to a target molecule can be determined by detecting the labeled protein in a complex. For example, proteins can be labeled with ¹²⁵I, ³⁵S, ¹⁴C, or ³H, either directly or indirectly, and the radioisotope detected by direct counting of radioemmission or by scintillation counting. Alternatively, molecules can be enzymatically labeled with, for example, horseradish peroxidase, alkaline phosphatase, or luciferase, and the enzymatic label detected by determination of conversion of an appropriate substrate to product.

Typically, it will be desirable to immobilize either a protein of the invention or its binding protein to facilitate separation of complexes from uncomplexed forms of one or both of the proteins, as well as to accommodate automation of the assay. Binding to an upstream or downstream binding element, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/Ian4 (GST/Ian4) fusion proteins can be adsorbed onto glutathione sepharose beads (Sigma Chemical, St. Louis, Mo.) or glutathione derivatized microtitre plates, which are then combined with the cell lysates, e.g. an ³⁵S-labeled, and the test modulating agent, and the mixture incubated under conditions conducive to complex formation, e.g., at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of Ian4-binding protein found in the bead fraction quantitated from the gel using standard electrophoretic techniques.

Other techniques for immobilizing proteins on matrices are also available for use in the subject assay. For instance, biotinylated molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with a protein of the invention, but which do not interfere with binding of upstream or downstream elements can be derivatized to the wells of the plate, and the protein trapped in the wells by antibody conjugation.

A variety of art recognized methods can be used to detect one or both of the proteins. For example, for processes which rely on immunodetection for quantitating one of the proteins trapped in the complex, antibodies against the protein, can be used. Alternatively, the protein to be detected in the complex can be “epitope tagged” in the form of a fusion protein which includes a portion of a second protein for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharamacia, NJ).

It is also within the scope of this invention to determine the ability of a compound to modulate the interaction between a protein of the invention and its target molecule, without the labeling of any of the interactants. For example, a microphysiometer can be used to detect the interaction of a protein of the invention with its target molecule without the labeling of either the protein or the target molecule. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

iii) Whole Organism Screens

The host cells of the invention can also be used to produce nonhuman transgenic animals. The nonhuman transgenic animals can be used in screening assays designed to identify agents or compounds, which are capable of ameliorating detrimental symptoms of selected disorders such as immune disorders and neoplasms. For example, in one embodiment, a host cell of the invention is a fertilized oocyte or an embryonic stem cell into which Ian4-coding sequences have been introduced. Such host cells can then be used to create non-human transgenic animals in which exogenous Ian sequences have been introduced into their genome or homologous recombinant animals in which endogenous Ian sequences have been altered. Such animals are useful for studying the function and/or activity of Ian family members and for identifying and/or evaluating modulators of Ian activity. As used herein, a “transgenic animal” is a nonhuman animal, preferably a mammal, more preferably a rodent such as a rat or mouse, in which one or more of the cells of the animal includes a transgene. Other examples of transgenic animals include nonhuman primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a nonhuman animal, preferably a mammal, more preferably a mouse, in which an endogenous Ian gene has been altered by homologous recombination between the endogenous gene and an exogenous DNA molecule introduced into a cell of the animal, e.g., an embryonic cell of the animal, prior to development of the animal.

A transgenic animal of the invention can be created by introducing Ian-encoding nucleic acid into the male pronuclei of a fertilized oocyte, e.g., by microinjection, retroviral infection, and allowing the oocyte to develop in a pseudopregnant female foster animal. The human Ian4 cDNA encoding the protein sequence of SEQ ID NO:2 can be introduced as a transgene into the genome of a nonhuman animal. Alternatively, a nonhuman homologue of the human Ian gene, such as a mouse Ian gene, can be isolated based on hybridization to the human Ian cDNA and used as a transgene. Intronic sequences and polyadenylation signals can also be included in the transgene to increase the efficiency of expression of the transgene. A tissue-specific regulatory sequence(s) can be operably linked to the Ian transgene to direct expression of Ian protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the Ian transgene in its genome and/or expression of Ian mRNA in tissues or cells of the animals. A transgenic founder animal can then be used to breed additional animals carrying the transgene. Moreover, transgenic animals carrying a transgene encoding Ian can further be bred to other transgenic animals carrying other transgenes.

To create a homologous recombinant animal, a vector is prepared which contains at least a portion of an Ian gene into which a deletion, addition or substitution has been introduced to thereby alter, e.g., functionally disrupt, the Ian gene. The Ian gene can be a human gene (e.g., human Ian4 nucleotide of SEQ ID NO:1), but more preferably, is a nonhuman homologue of a human IAN gene. For example, a mouse Ian gene can be isolated from a mouse genomic DNA library using the human Ian cDNA encoding the human Ian4 protein of SEQ ID NO:1 as a probe. The mouse Ian gene then can be used to construct a homologous recombination vector suitable for altering an endogenous Ian gene in the mouse genome. In a preferred embodiment, the vector is designed such that, upon homologous recombination, the endogenous Ian gene is functionally disrupted (i.e., no longer encodes a functional protein; also referred to as a “knock out” vector). Alternatively, the vector can be designed such that, upon homologous recombination, the endogenous Ian gene is mutated or otherwise altered but still encodes functional protein (e.g., the upstream regulatory region can be altered to thereby alter the expression of the endogenous Ian protein). In the homologous recombination vector, the altered portion of the Ian gene is flanked at its 5′ and 3′ ends by additional nucleic acid of the Ian gene to allow for homologous recombination to occur between the exogenous Ian gene carried by the vector and an endogenous Ian gene in an embryonic stem cell. The additional flanking Ian nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced Ian gene has homologously recombined with the endogenous Ian gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic nonhumans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the nonhuman transgenic animals described herein can also be produced according to the methods described in Wilmut, I. et al. (1997) Nature 385:810-813. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated.

Non-human transgenic animals of the invention can be used in whole organism screens to identify modulatory agents that alter the expression and/or activity of the proteins of the invention. In one embodiment, a non-human transgenic animal containing a functionally disrupted gene of the invention that produced an observable mutant phenotype (e.g. lymphopenia) can be used in whole-organism assays to identify modulatory agents that functionally complement or rescue the mutant phenotype. Alternatively, established animal models wherein mutant forms of the genes of the invention have arisen spontaneously or by artificial mutagenesis (e.g. BB Diabetes Prone rats containing the Ian4 frameshift mutation) can be utilized in screening assays to identify modulatory agents that functionally compelement or rescue the mutant phenotype.

This invention further pertains to novel agents identified by the above-described screening assays. Accordingly, it is within the scope of this invention to further use an agent identified as described herein in an appropriate animal model. For example, a modulating agent identified as described herein (e.g., an Ian4 modulating agent, an antisense Ian4 nucleic acid molecule, an Ian4-specific antibody, or an Ian4-binding partner) can be used in an animal model to determine the efficacy, toxicity, or side effects of treatment with such an agent. Alternatively, an agent identified as described herein can be used in an animal model to determine the mechanism of action of such an agent. The efficacy of the modulating agent can be assessed by generating dose response curves from data obtained using various concentrations of the test modulating agent. Moreover, a control assay can also be performed to provide a baseline for comparison. Furthermore, this invention pertains to uses of novel agents identified by the above-described screening assays for treatments as described herein.

This invention is further illustrated by the following examples which should not be construed as limiting. The contents of all references, patents and published patent applications cited throughout this application are hereby incorporated by reference. Nucleotide and amino acid sequences deposited in public databases as referred to herein are also hereby incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXEMPLIFICATION

The present invention is further illustrated by the following examples which should not be construed as limiting in any way.

The following materials and methods were used in the Examples:

Animals. BBDR and BBDP rats were obtained from BMR, Inc. (Worcester, Mass.). About 90% of BBDP rats develop spontaneous autoimmune diabetes; they are Ian4^{-/-} and congenitally lymphopenic. BBDR rats are Ian4^{+/+} and never become spontaneously diabetic (Mordes, J. P. et al. Animal models of diabetes: A primer. Sima, A. A. F. & Shafrir, E. (eds.), pp. 1-41 (Harwood Academic Publishers, Amsterdam, 2001). WF rats were obtained from Harlan Sprague Dawley (Indianapolis, Ind.). A congenic, non-diabetic WF.ART2a rat bred in our laboratories was used in one experiment (Mordes, J. P. et al. The iddm4 locus segregates with diabetes susceptibility in congenic WF.iddm4 rats. Diabetes 51, 3254-3262 (2002)) Eight to 10 week old rats of either sex were used; all rats were non-diabetic at the time of study. Animals were housed in a viral-antibody free facility and maintained in accordance with the Guide for the Care and Use of Laboratory Animals (National Academy of Sciences, 1996) and guidelines of our Institutional Animal Care Committee.

Antibodies

Purified and fluorochrome- or biotin-conjugated monoclonal antibodies (mAbs) directed against CD3 (G4.18), CD28 (JJ319), and αβTCR(R7.3) were obtained from BD PharMingen (San Diego, Calif.). Isotype control mAb mouse IgG1 and APC®-conjugated streptavidin were also purchased from BD PharMingen. Anti-actin and anti-cytochrome C antibodies were from Chemicon International, Temecula, Calif. and BD Pharmingen, San Diego, Calif., respectively. Anti-rabbit and anti-mouse IgG horseradish peroxidase (HRP) conjugate was obtained from Santa Cruz Biotechnology (Santa Cruz, Calif.) or Promega (Madison, Wis.).

Cell Isolation

Rats were killed in an atmosphere of 100% CO₂ and cervical and mesenteric lymph nodes, thymi, and liver were removed. Single cell suspensions were prepared by gentle extrusion through stainless steel sieves into medium consisting of cold RPMI-1640 supplemented with 5% Fetalclone® (Gibco BRL, City) as described (N. N. Iwakoshi et al., J. Immunol. 160, 5838-5850 (1998)). To obtain purified T cell preparations, lymph node cells were passed over nylon wool columns (Polysciences, Inc., Warrington, Pa., USA). In some experiments, purified T and B cells were prepared using magnetic bead technology according to the manufacturer's directions (Miltenyi Biotec, Auburn Calif.). Briefly, antibody-labeled cells were incubated for 20 min with rat anti-mouse IgG1 Microbeads (Miltenyi Biotec) at 4° C. After two washes, bound cells were separated using appropriate columns placed in a strong magnetic field (MidiMACS, Miltenyi Biotec). Peritoneal exudate macrophages were obtained from animals injected intraperitoneally with 3 ml 3% thioglycollate (Sigma, St. Louis, Mo.); macrophages were harvested from the peritoneal cavity 4 days after injection.

Mitochondrial Isolation

T lymphocytes and thymocytes were centrifuged in complete AIM-V medium (AIM-V plus 55 μM 2-mercaptoethanol, Sigma) at 1000 rpm for 5 min in a Beckman GPKR centrifuge. Medium was removed and cells washed in medium A (medium A: 100 mM sucrose, 1 mM EGTA, 20 mM Hepes, pH 7.4, 1 g/L BSA) 3 times, centrifuging at 1000 rpm at for 5 min between each wash. Cells were resuspended in 1 ml medium B (medium B: medium A plus 10 mM triethanolamine, 5% Percoll, 0.1 mg/ml digitonin) and incubated for 3 min at 4° C. The cells were homogenized with 10 strokes of a motor driven (500 rpm) glass-Teflon pestle. The homogenate was centrifuged at 2500×g for 5 min and the supernatant removed. The pellet was resuspended in 1 ml medium B and homogenized as described above. The homogenate was centrifuged at 2500×G for 5 min. The supernatant was removed and the pellet was resuspended in 20% sucrose, 10 mM Tris and 0.1 mM EDTA and centrifuged at 18,000×g for 30 min. The pellet was resuspended in 60% sucrose, 10 mM Tris and 0.05 mM EDTA. The suspension was layered with 53% sucrose, 10 mM Tris and 0.05 mM EDTA, which was layered with 44% sucrose, 10 mM Tris and 0.05 mM EDTA. The sucrose step gradient was centrifuged at 141,000×G for 2 h. Purified mitochondria settled at the 44/53 interface. The mitochondrial layer was removed, diluted in medium A without BSA and centrifuged at 18,000×G for 30 min. The pellet was resuspended in 50 μl of medium A without BSA. 120 mM CHAPS was added to the suspension to a final concentration of 6 mM and vortexed. Medium A without BSA was added to the suspension to a final CHAPS concentration of 3 mM, vortexed continuously for 10 min and the protein concentration measured by the Bradford assay (Bio-Rad Laboratories, Hercules, Calif.). Equal concentrations of protein were loaded onto 1.9 ml 10-30% sucrose gradients and centrifuged at 35,000 rpm in a TLS55 rotor (Beckman Instruments, Fullerton, Calif.) for 20.33 hr. Each gradient was fractionated into 10×200 μl fractions from the top and analyzed by SDS-PAGE. Gels were analyzed using either Western blotting or Sypro® Ruby protein stain (Molecular Probes, Eugene, Oreg.) as described below.

Western Blot Analysis

Single cell suspensions were isolated as described above and incubated in lysis buffer (150 mM NaCl, 50 mM HEPES, pH 7.6, 1% Brij 96, 2 mM EDTA, 1 mM benzamide, 1 mM PMSF, 1 mM NaVO₄, 20 mM NaF, leupeptin, 10 μg/ml, and antipain, 10 μg/ml) at 4° C. for 30 to 60 min. Lysates were centrifuged (8000×G, 10 min) to pellet insoluble material. Supernatants (20 μl) were added to and equal volume of SDS-loading buffer (20% glycerol, 6% 2-mercaptoethanol, 1.5% SDS, 62.5 mM Tris pH 6.8, 0.1% bromphenyl blue) and then boiled for 5 min. Proteins were separated on a 12% SDS-polyacrylamide gel and transferred to PVDF membranes. Membranes were incubated overnight at 4° C. with 5% non-fat dry milk (Bio-Rad) in Tris-buffered saline (TBS, pH 7.6) and then incubated for 1 h at room temperature with appropriately diluted reagents in TBS containing 1% non-fat dry milk. Membranes were washed with TBS, and incubated with peroxidase-conjugated secondary antibody (goat anti-rabbit IgG with peroxidase, Boehringer Mannheim, Indianapolis, Ind.) in TBS. After three 10 minute washes with TBS, the immunoblots were developed using enhanced chemiluminescence (ECL) Western blotting detection reagents (Amersham, Piscataway, N.J.) and exposed to Kodak X-Omat AR film.

In Vitro T Cell Stimulation

Flat bottom 6-well culture plates (Falcon, Franklin Lakes, N.J.) were incubated with 10 μg/well anti-CD3 mAb, 10 μg/well anti-CD28 mAb, or both, in PBS at 4° C. After overnight incubation, plates were rinsed three times with PBS. Nylon wool-purified T cells were cultured in antibody-coated wells at 6×10⁶ cells/well in 3 ml complete AIM-V media (AIM-V plus 55 μM 2-mercaptoethanol, Sigma) for 17, 40, or 68 hr in a humidified incubator at 37° C. with 5% CO₂. Un-stimulated cells were incubated in complete AIM-V alone.

Sub-Diploid DNA and Cell Cycle Analysis

The percentage of T cells with sub-diploid DNA was measured as described (I. Nicoletti, G. Migliorati, M. C. Pagliacci, F. Grignani, C. Riccardi, J. Immunol. Methods 139, 271-279 (1991)). Briefly, freshly isolated or in vitro stimulated lymph node T cells were washed twice with PBS and centrifuged at 1500 rpm for 5 min. Pellets (2×10⁶) were resuspended in 200 μl of PBS, fixed in 70% cold ethanol, and stored at 4° C. until used. At the time of analysis, cells were washed twice with PBS and incubated at 25° C. for 40 min in 500 μl of PBS containing 0.1% Triton X-100 and 50 μg of RNase (Boehringer Mannheim, Indianapolis, Ind.). Propidium iodide (10 μg, Sigma, St. Louis, Mo.) was added, and the suspension was incubated in the dark for 15 min. DNA content was determined by flow microfluorometry using a FACStarR® instrument (Becton Dickinson, San Jose, Calif.). Lymphoid cells were identified electronically by their forward and side light-scatter characteristics. For each analysis, a minimum of 10,000 events was analyzed.

Determination of Mitochondrial Membrane Potential (Δψₘ)

Mitochondrial membrane potential (Δψₘ) of thymocytes and T cells was measured by two- or three-color flow microfluorometry using MitoLight™ (Chemicon, Temecula, Calif.) alone or in combination with biotin-conjugated anti-αβTCR mAb. Mitolight™ was employed according to the manufacturer's instructions. It is a lipophilic cation with a cellular distribution similar to that of JC-1. JC-1 is a hydrophobic carbocyanine compound with delocalized positive charge that allows negative potential to drive its cellular distribution. When the concentration of JC-1 reaches a threshold, it forms aggregate, causing a shift in its absorbed and emitted fluorescence (S. T. Smiley et al., Proc. Natl. Acad. Sci. U.S.A 88, 3671-3675 (1991)). Briefly, cell suspensions were adjusted to 1×10⁶ cells/ml and incubated with 0.5 μg/ml Mitolight™ in medium supplied by the manufacturer for 15 minutes at 37° C. in the dark. Cells were washed twice and suspended in the supplied buffer. In some experiments designed to analyze T cells only, 1×10⁶ viable cells were first incubated with biotin-conjugated anti-αβTCR mAb for 30 min on ice. Cells were then washed and incubated for an additional 30 minutes on ice with APC-conjugated streptavidin to visualize biotinylated mAb. For each experiment control and BBDP T cells were analyzed for both red and green fluorescence after Mitolight™ labeling. As a positive control for dissipation of Δψₘ, cells were incubated for 15 minutes in AIM-V medium at 37° C. with the uncoupling reagent, carbonyl cyanide m-chlorophenyl-hydrazone (CCCP, 50 μM, Sigma), a protonophore that disrupts Δψₘ. To detect CSA mediated inhibition of Δψₘ, cells were cultured with Cyclosporin A (Sigma, 4 μM in DMSO) for 30 minutes at 37° C. Lymphoid cells were identified electronically by their forward and side light-scatter characteristics. For each analysis, a minimum of 10,000 events was analyzed.

High Resolution Fluorescence Microscopy

Nylon wool purified BBDP and BBDR lymph node T cells were placed on glass coverslips in a 37° C. heated chamber in the presence of MitoLight™ mitochondria-specific dye (0.5 μg/ml, Chemicon, Temecula, Calif.). Images were acquired with a Nikon 60× Plan-Apo, N.A. 1.4, with the dye excited and recorded with a Texas red filter cube (treated as Texas red). Images of each field were acquired as a 3D stack of ˜16 wide-field “optical sections” separated by 0.5 micron in the z (focus) dimension with a pixel size at the specimen of 0.166 micron. The stacked image file was then processed by deconvolution, deblurring. Data from a 3D stack of ˜16 sections were then projected onto a single plane for the resulting 3D projections, which show the general morphology of the cells.

Mass Spectrometry

Proteins resolved by SDS-PAGE were visualized by Sypro® Ruby protein stain. Bands were excised from the polyacrylamide gel and tryptically digested in-gel. The samples were analyzed by matrix-assisted laser desorption ionization-time of flight mass spectrometry (MALDI-TOF). For these analyses, digested samples were concentrated and desalted with Millipore Zip Tip C18 microtips (Millipore Corp., Bedford, Mass.). Peptide masses were determined using a Kratos Analytical Axima CFR MALDI-TOF spectrometer equipped with a curved field reflectron. Peptide masses were searched against the non-redundant protein database using MS-Fit of the Protein Prospector program available at http “donatello.ucsf.edu”. Fragmentation data from individual peptides via postsource decay analysis were searched against the non-redundant protein database using the Protein Prospector MS-Tag routine.

RNA Interference Assays

RNA interference assays were performed using sub-confluent Jurkat cells (ATCC, Rockville, Md.). The cells were plated onto flat bottom 12-well culture plates (Falcon 3043) at a density of 3×10⁵ cells per well in 1 ml 10% FBS in RPMI. A predicted human Ian4 oligonucleotide (AAGGTGAAAGAGGTCTTTGGG) (SEQ ID NO: 11) was synthesized, purified, and duplexed by Xeragon Inc. (Huntsville, Ala.). For use as a negative control, the duplex siRNA (AATTCTCCGAACGTGTCACGT) (SEQ ID NO: 12) was designed and synthesized by Xeragon. The lyophilized siRNA was suspended in a supplied buffer according to the manufacturer's instructions. The transfection vehicle Trans-TKO (6 μl, Mirus, Madison, Wis.) was added to aliquots of RPMI (50 μl) and incubated for 15 min at room temperature, after which siRNA was added. After mixing gently for 20 min at room temperature, aliquots containing Ian4 siRNA, the control siRNA, or the transfection vehicle alone were added to each well. The final concentration of siRNA in each well was either 250 nM or 500 nM. Cells were then incubated for 48 hrs at 37° in a humidified atmosphere of 95% air, 5% CO₂. At the end of the incubation, the percentage of cells with sub-diploid DNA was determined as above.

Caspase inhibition

Lymph node T cells from BBDP rats were prepared as described above and cultured in AIM-V medium in the presence or absence of varying concentrations of the caspase inhibitors Z-VAD-FMK, Z-VDVAD, Z-DEVD, D-IETD, and Z-LEHD obtained from R&D Systems (Minneapolis, Minn.). Inhibitors were used according to manufacturer's instructions. Cells were cultured for 17 hr at 37° in a humidified atmosphere of 95% air 5% CO₂. Cells were then harvested, and the percentage of cells with sub-diploid DNA was determined as above.

Statistics

Parametric data are presented as arithmetic means ±1 SD. Groups of 3 or more means were compared by oneway analysis of variance and the least significant differences procedure for a posteriori contrasts (N. H. Nie, C. H. Hull, J. G. Jenkins, K. Steinbrenner, D. H. Bent, Statistical package for the social sciences (McGraw-Hill, New York, ed. 2, 1975), pp. 1-675). Pairs of means were compared using two-tailed t-tests with separate variance estimates and the Bonferroni adjustment for multiple comparisons was applied as necessary (S. A. Glantz, Primer of biostatistics (McGraw-Hill, New York, 1981), p. 352).

### EXAMPLE 1

### Generation of an Anti-Ian4 Antibody

Polyclonal antiserum to detect Ian4 was generated against a KLH-conjugated peptide translated from GenBank accession number AC099444.2 that corresponds to residues 152-173 of rat Ian4S protein (AAL17698). This sequence would be absent from any truncated form of Ian4 that might have been translated in the BBDP rat (L. Hornum, J. Romer, H. Markholst, Diabetes 51, 1972-1979 (2002)).

### EXAMPLE 2

### Ian4 is Expressed in Thymocytes, Lymph Node T Cells and B Cells

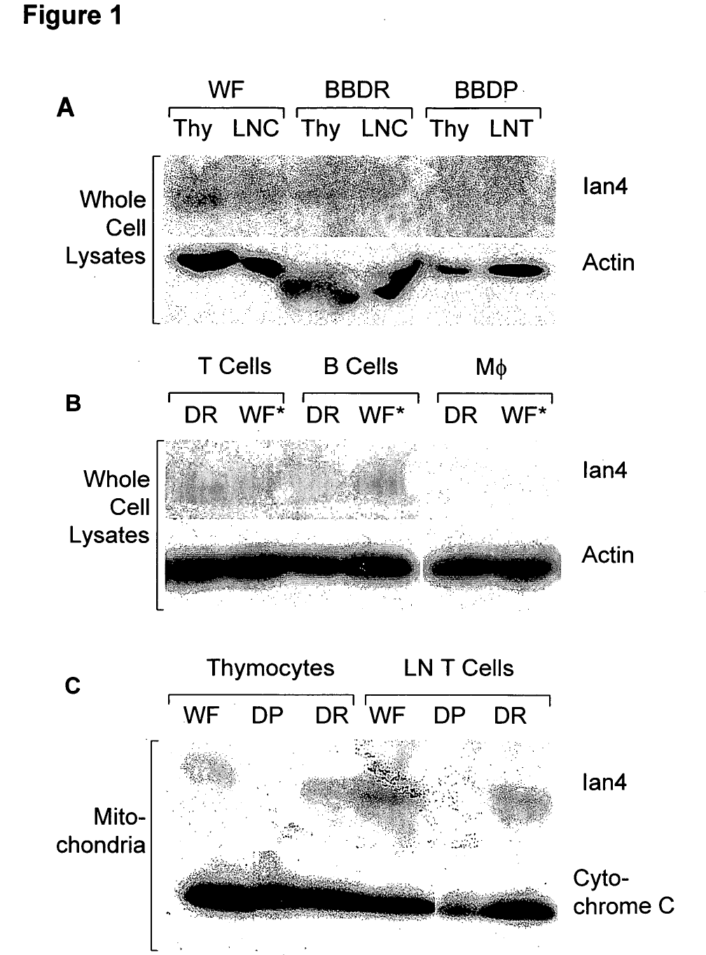

Lymphoid cells from non-diabetic Ian4^{-/-} BBDP rats (Hornum, L., et al. Diabetes 51, 1972-1979 (2002); MacMurray, A. J. et al. Genome Res. 12, 1029-1039 (2002); Mordes, J. P. et al. Animal models of diabetes: A primer. Sima, A. A. F. & Shafrir, E. (eds.), pp. 1-41 (Harwood Academic Publishers, Amsterdam, 2001)) were compared with those isolated from control, non-lymphopenic WF and BBDR rats. The Ian4 frameshift mutation in the BBDP rat generates a truncated protein product that lacks the COOH— terminal transmembrane region required for mitochondrial membrane localization Hornum, L., Rømer, J. & Markholst, H. The diabetes-prone BB rat carries a frameshift mutation in Ian4, a positional candidate of iddml. Diabetes 51, 1972-1979 (2002)). Western blotting of Ian4^{+/+} BBDR and WF cell lysates with a polyclonal antibody to Ian4 revealed a ˜30 kD band in thymocytes, lymph node T cells, and B cells but not macrophages (FIGS. 1A and B). Panel A shows thymocyte (Thy) and lymph node cell (LNC) lysates from Ian4^{+/+} BBDR (“DR”) and WF and Ian4^{-/-} BBDP (“DP”) rats. Panel B shows lymph node T cell, splenic B cell, and macrophage lysates from Ian4^{+/+} BBDR and WF.ART2a rats (Mordes, J. P. et al. The iddm4 locus segregates with diabetes susceptibility in congenic WF.iddm4 rats. Diabetes 51, 3254-3262 (2002)). Panel C shows purified mitochondrial lysates from T cells and thymocytes of Ian4^{+/+} BBDR and WF and Ian4^{-/-} BBDP rats. Actin and cytochrome C were used as loading controls. No Ian4 was detected in lymphoid tissues of Ian4-” BBDP rats. Western blots also demonstrated Ian4 protein in purified mitochondria obtained from thymocytes and T cells of Ian4^{+/+} but not Ian4^{-/-} rats (FIG. 1C).

### EXAMPLE 3

### Ian4 Promotes Mitochondrial Membrane Integrity

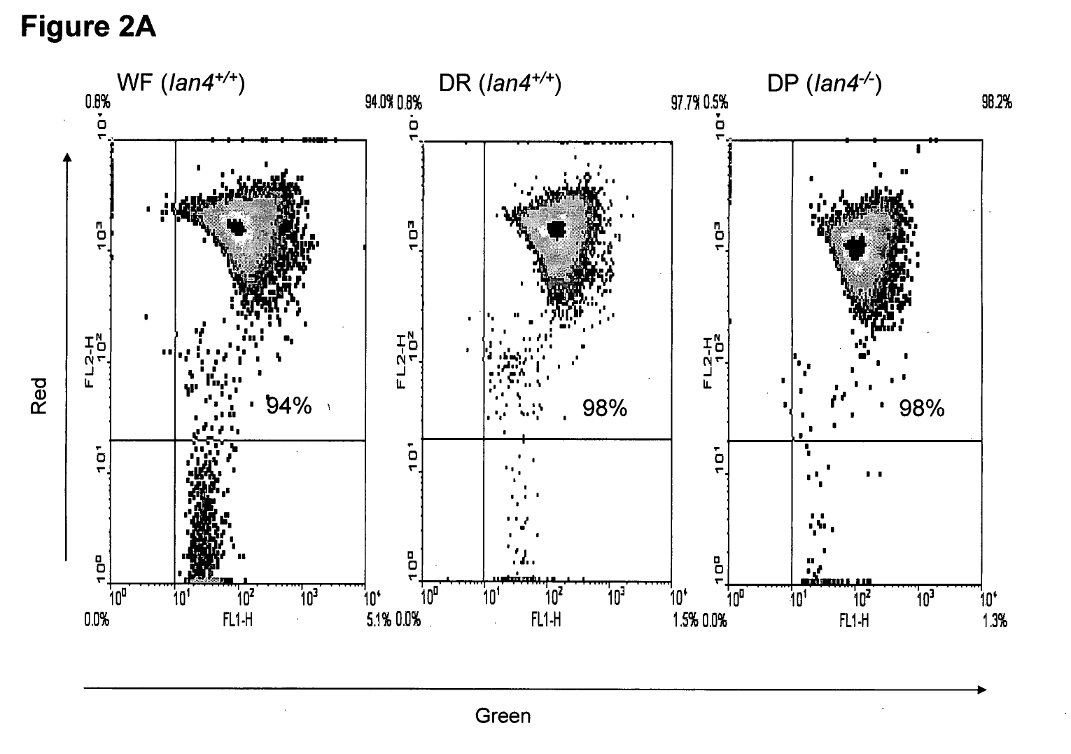

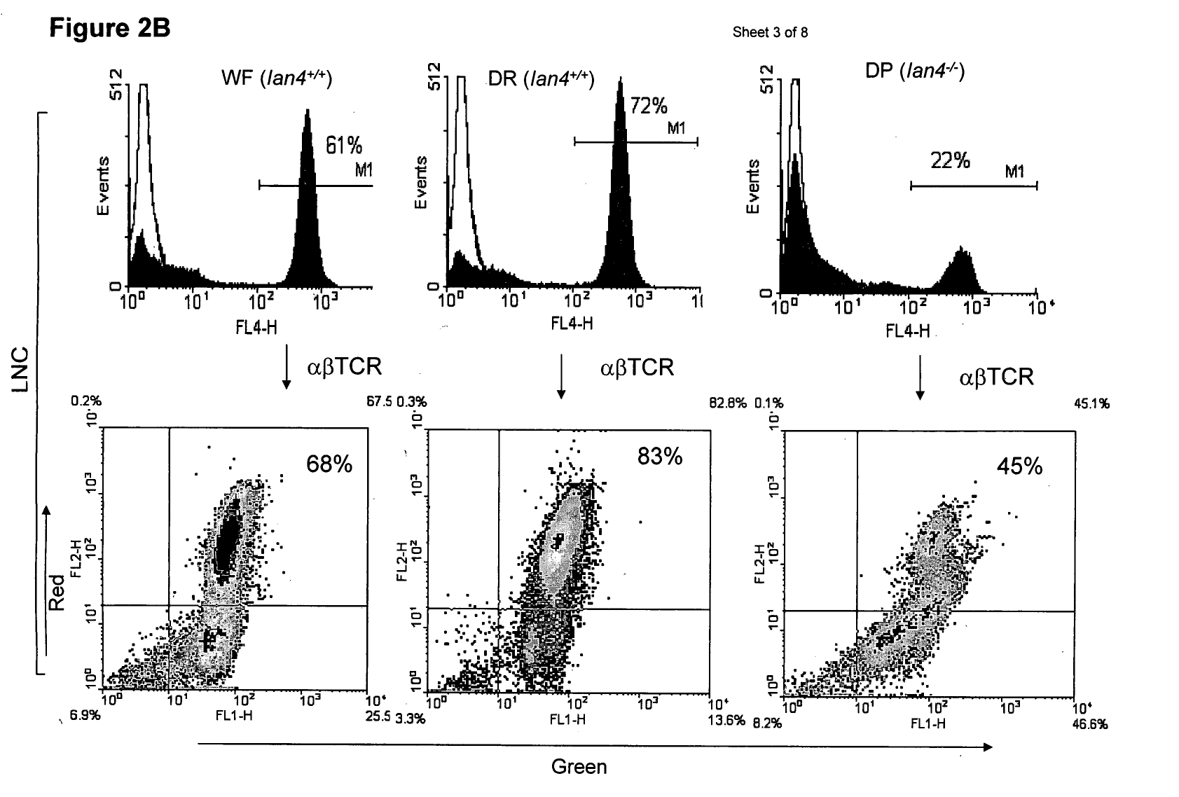

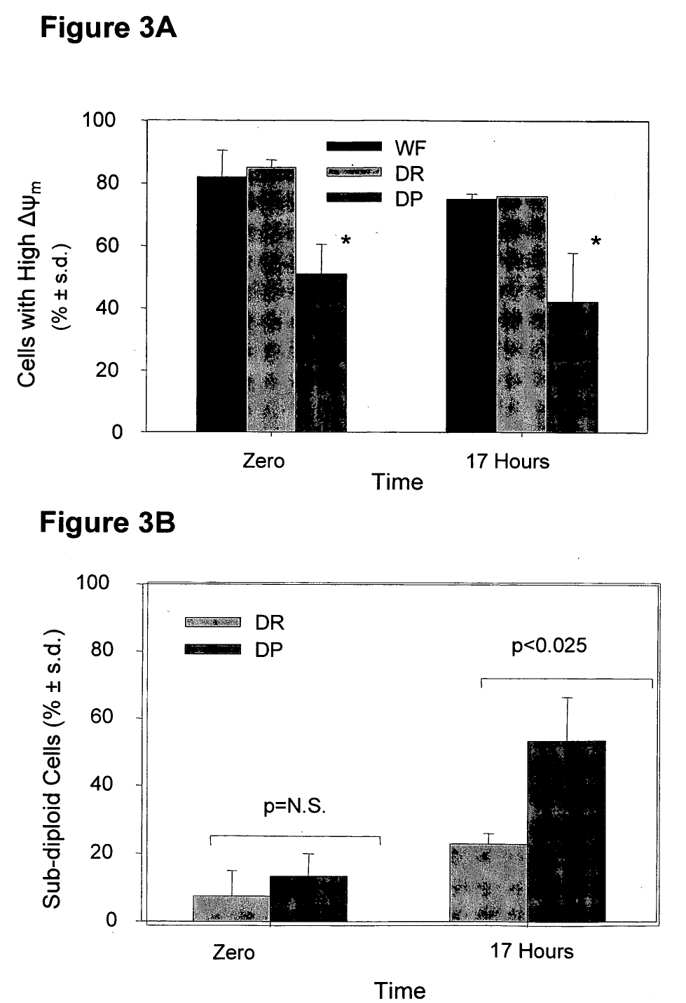

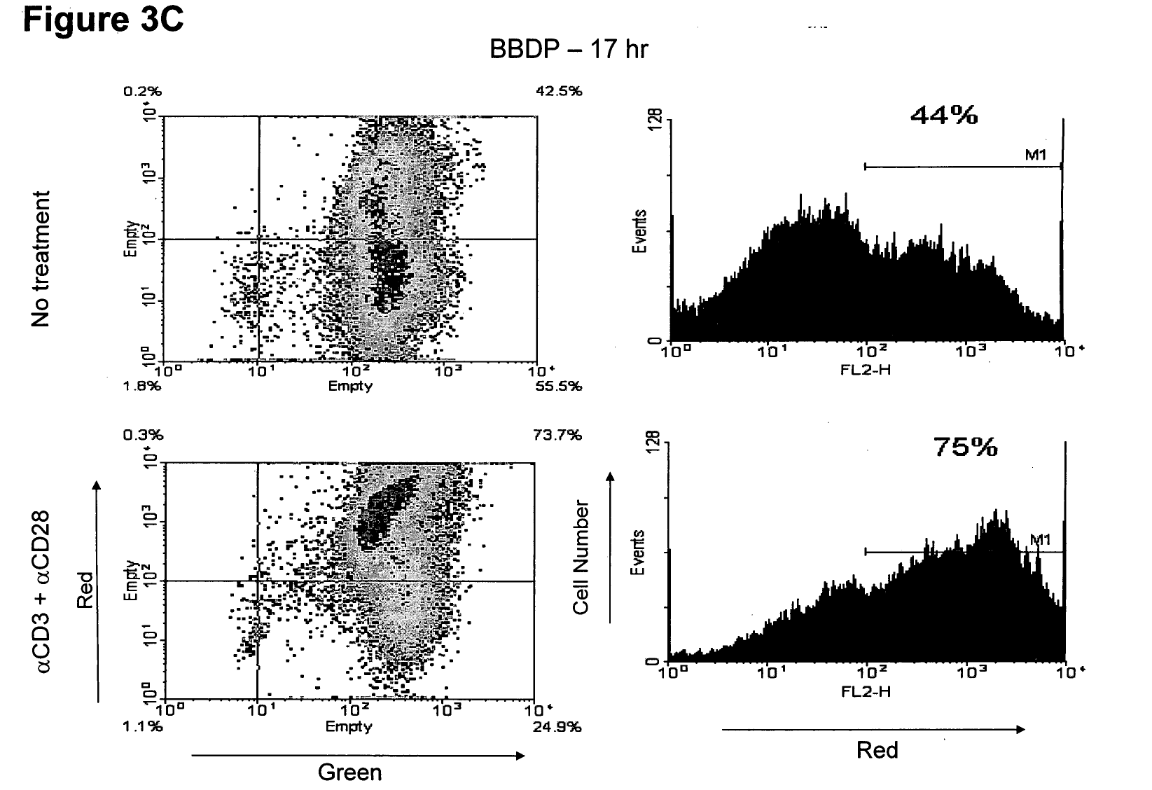

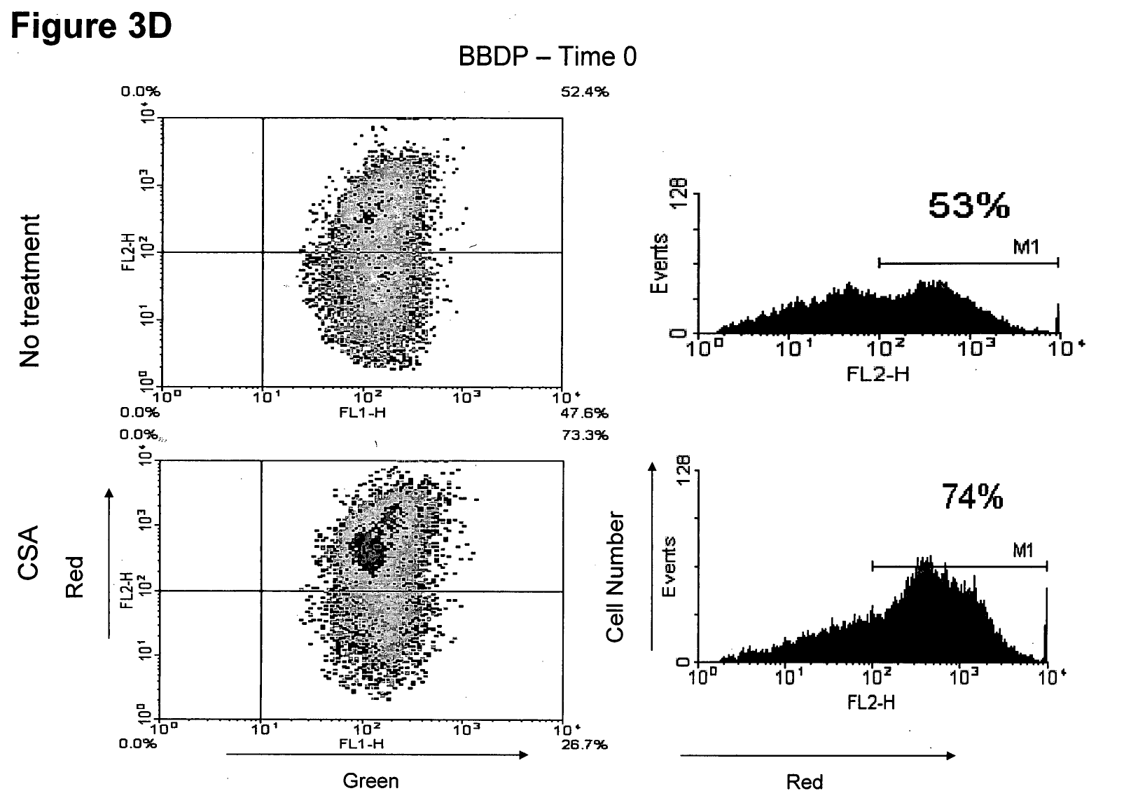

Mitochondrial membrane potential (Δψₘ) was measured in wild-type and Ian4^{-/-} T cells using a mitochondrion-specific dye, Mitolight™, which displays a green to red spectral shift proportional to Δψₘ (Smiley, S. T. et al. Proc. Natl. Acad. Sci. U.S.A 88, 3671-3675 (1991)). High resolution digital fluorescence microscopy of cells from both Ian4^{-/-} BBDP and Ian4^{+/+} BBDR rats confirmed that the dye labels mitochondrial structures, with no detectable staining of other organelles. Mitochondrial structure appeared similar in T cells from Ian4^{-/-} and Ian4^{+/+} rats. Panel A of FIG. 2 shows Δψₘ of thymocytes from Ian4^{+/+} (WF, BBDR) and Ian4^{-/-} (BBDP) rats; shown are the relative percentages of cells fluorescing red (vertical axis, high ΔΨₘ) and green (horizontal axis, low Δψₘ). The majority of thymocytes from both Ian4+/+ and Ian4^{-/-} rats display high Δψₘ. Data are representative of two independent experiments. Panel B shows Δψₘ of lymph node T cells. Horizontal bars in histograms indicate gates used to identify αβTCR⁺ cells. The T lymphopenia of BBDP rats is evident. The dot plots show that the percentage of T cells with high Δψₘ was lower in Ian4^{-/-} than in Ian4^{+/+} rats. Data are representative of three independent experiments; the complete dataset is shown in FIG. 3A. Control incubations with the mitochondrial uncoupler CCCP uniformly reduced the percentage of T cells with high Δψₘ to ˜4%. Quantitative analysis revealed that Δψₘ was similar and normal in freshly isolated thymocytes from all three rat strains (FIG. 2A) but substantially lower in freshly isolated resting T cells from Ian4^{-/-} BBDP rats than in T cells from Ian4^{+/+} rats (FIGS. 2B and 3A). Panel A of FIG. 3 shows peripheral T lymphocytes from Ian4^{+/+} (WF and BBDR) rats and Ian4^{-/-} (BBDP) rats were assayed for Δψₘ at time of isolation (Time Zero) and after 17 hrs of culture. Shown are results of three independent experiments analyzed by ANOVA (*p<0.003 at time 0 and *p<0.006 at 17 hrs vs. BBDR and WF). Panel B shows the percentage of cells with sub-diploid DNA in peripheral T lymphocytes from BBDR and BBDP rats was determined by propidium iodide staining and flow microfluorometry when isolated (Time Zero) and after 17 hrs of culture. Results shown are from three independent experiments. Panels C and D show Ian4^{-/-} BBDP T cells incubated for 17 hrs with anti-CD3 plus anti-CD28 mAbs (C) or for 30 min with cyclosporin A (D). Gates used to identify the percentage of cells with high Δψₘ are indicated by horizontal bars. Data are representative of three independent experiments. Both activation and cyclosporin A rescued Ian4^{-/-} lymphocytes from loss of Δψₘ. The result suggests that loss of ΔΨₘ in Ian4^{-/-} BBDP rat T cells occurs after thymic export. Kinetic analysis showed that the percentage of freshly isolated T cells with high Δψₘ did not change significantly in any of the strains during 17 hours of culture (FIG. 3A). In contrast, the percentage of sub-diploid (apoptotic) lymph node T cells immediately after isolation was similar in Ian4^{-/-} and wild-type rats (FIG. 3B), but within 17 hours became significantly higher in Ian4^{-/-} animals.

The apoptosis of T cells from BBDP rats (FIG. 3B) is reportedly prevented by activation (Ramanathan, S., Norwich, K. & Poussier, P. J. Immunol. 160, 5757-5764 (1998)) Activation in vitro normalized ΔΨₘ in these Ian4^{-/-} T cells (FIG. 3C). The result suggests that loss of Δψₘ is an early event in the pathway leading to spontaneous apoptosis in Ian4^{-/-} T cells.

Maintenance of Δψₘ depends on mitochondrial membrane integrity and proper regulation of the permeability transition pore (PTP) (Scorrano, L., Nicolli, A., Basso, E., Petronilli, V. & Bemardi, P. Mol. Cell Biochem. 174, 181-184 (1997)). The ability of Ian4 to help maintain normal PTP function was tested. Ian4^{-/-} BBDP T cells were incubated in the presence of cyclosporin A, which prevents mitochondrial membrane pore transition (Scorrano, L., Nicolli, A., Basso, E., Petronilli, V. & Bernardi, P. Mol. Cell Biochem. 174, 181-184 (1997)). Treatment of Ian4^{-/-} T cells with cyclosporin A was associated with a rapid increase in the percentage of T cells with high Δψₘ (FIG. 3D), suggesting that Ian4 may be involved in the regulation of the PTP.

### EXAMPLE 4

### Removal of Ian4 Induces Apoptosis

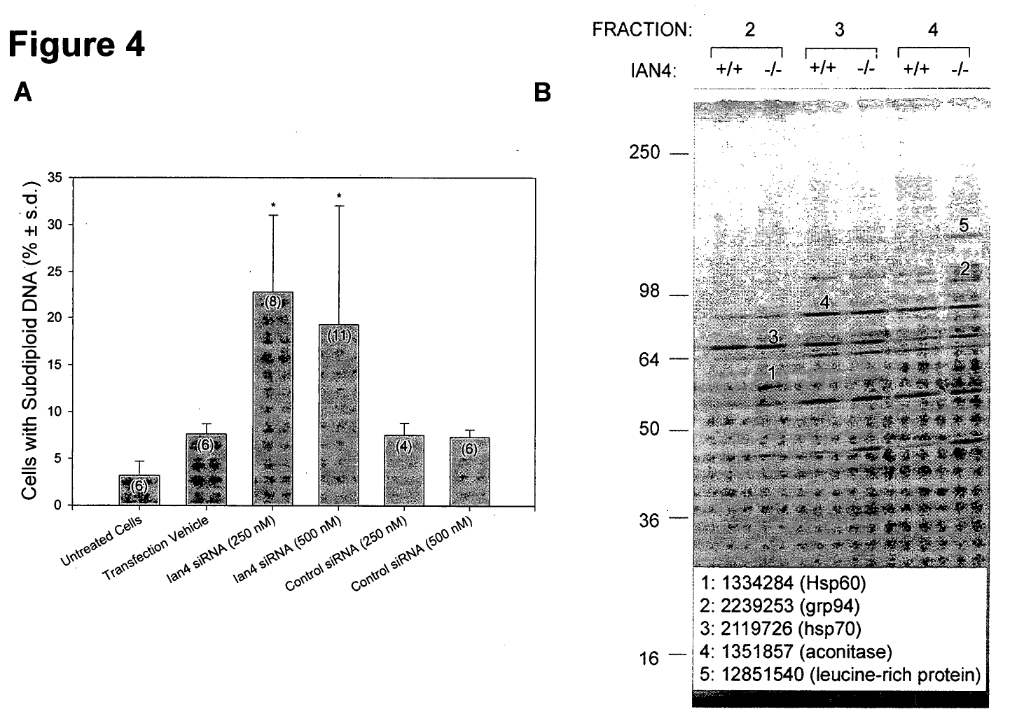

If Ian4 expression is required to prevent spontaneous apoptosis in T cells, its removal should induce apoptosis. This inference was confirmed using human Jurkat T cells. Transfection with siRNA specific to Ian4 increased the percentage of T cells with sub-diploid DNA, an effect not observed in non-transfected cells or in cells treated with transfection reagent alone or with non-specific control siRNA (FIG. 4A). Panel A of FIG. 4 shows transfection of Ian4-specific siRNA increased T cell apoptosis. Bars depict the percentage of cells with sub-diploid DNA after 48 hr of treatment. *Statistically similar to each other and p<0.025 vs. each of the other 4 groups. The number of independent measurements is shown in parenthesis. Two other Ian4 siRNAs were less effective. Impairment of Ian4 translation in Jurkat T cells thus reproduces the spontaneous apoptosis of Ian4^{-/-} BBDP T cells, the apoptotic phenotype of which is likely to be due specifically to loss of Ian4.

### EXAMPLE 5

### Expression of Certain Proteins is Enhanced in Ian−/− Cells

The protein profiles of purified mitochondria from BBDP, BBDR, and WF cells were then compared. Analysis by velocity gradient centrifugation (Wilson-Fritch, L. et al. Mol. Cell. Biol. 23, 1085-1094 (2003)) revealed proteins of ˜60, 100, and 130 kD that were over-represented in mitochondrial fractions from Ian4^{-/-} T cells (FIG. 4B). Panel B of FIG. 4 shows purified mitochondrial proteins from Ian4^{+/+} WF and Ian4^{-/-} BBDP rat T cells resolved by SDS-PAGE. Bands 1, 2, and 5 were differentially expressed in Ian4^{+/+} vs. Ian4^{-/-} T cells, excised, and identified by MALDI-TOF mass spectrometry. Not all differentially expressed bands were examined. Bands 3 and 4, which were similarly expressed, were excised to verify the purity of the mitochondrial isolation. Identities and gene identifiers for excised bands are indicated. Shown is one of two similar gels.

No proteins were over-represented in Ian4^{-/-} thymus or liver mitochondrial extracts. MALDI-TOF mass spectrometric analysis identified the ˜60 and ˜100 kD proteins as the stress-inducible proteins hsp60 and GRP94, respectively (FIG. 4B). The ˜130 kD protein was very similar to a putative mouse protein of unknown function with a predicted mass of 130 kD that is highly homologous to a human 130 kD leucine-rich protein upregulated in the HepG2 hepatoblastoma cell line (Hou, J., Wang, F. & McKeehan, W. L. In Vitro Cell Dev. Biol. Anim 30A, 111-114 (1994)). Mitochondrial proteins unaffected by the presence or absence of Ian4 included aconitase and hsp70 (FIG. 4B).

### EXAMPLE 6

### Apoptosis in the Absence of Ian4 Involves Caspases

These data suggest that absence of an Ian4-dependent signal in T cell mitochondria leads to indolent activation of intrinsic apoptosis pathways after thymic emigration. As many apoptotic pathways involve caspase activity, a general inhibitor of caspases, Z-VAD-frnk, and specific inhibitors of caspases 2, 3, 8, and 9 in Ian4^{-/-} T cells were tested. These four caspases reportedly exist as pro-enzymes in the mitochondrial intermembrane space (Parone, P. A., James, D. & Martinou, J. C. Biochimie 84, 105-111 (2002)) and are released and activated by apoptotic stimuli (Qin, Z. H. et al. Pro-caspase-8 is predominantly localized in mitochondria and released into cytoplasm upon apoptotic stimulation. J. Biol. Chem. 276, 8079-8086 (2001)). Both the general caspase inhibitor and inhibitors of caspases 3 and 8 prevented apoptosis of Ian4^{-/-} T cells after 17 hr of culture. Inhibitors of caspases 2 and 9 were much less effective. The apparent lack of involvement of caspase 9 suggests that Ian4-mediated apoptosis is different from the classical intrinsic pathway or possibly that Ian4 is an unrecognized component of the extrinsic pathway, consistent with the ability of caspase 8 inhibitors to prevent apoptosis.

Equivalents

Those skilled in the art will recognize, or be able to ascertain using no more than routine experimentation, many equivalents to the specific embodiments of the invention described herein. Such equivalents are intended to be encompassed by the following claims.

1. A method of specifically modulating apoptosis in a cell of lymphoid origin, comprising contacting the cell with a compound that modulates the expression or activity of an Ian-related gene or protein, such that apoptosis in the cell is specifically modulated.

2. The method of claim 1, wherein the Ian gene or protein is Ian4.

3. The method of claim 1, wherein the cell is a T cell.

4. The method of claim 3, wherein the cell is a CD4+ cell.

5. The method of claim 3, wherein the cell is a CD8+ cell.

6. The method of claim 1, wherein apoptosis is downmodulated.

7. The method of claim 1, wherein apoptosis is upmodulated.

8. The method of claim 1, wherein the cell has previously received a signal that induces apoptosis.

9. The method of claim 1, wherein the compound modulates mitochondrial permeabilization.

10. The method of claim 9, wherein the compound modulates inner mitochondrial membrane permeabilization.

11. The method of claim 1, wherein the compound modulates the release of apoptogenic factors from mitochondria within the cell.

12. The method of claim 11, wherein the apoptogenic factor is procaspase-2, procaspase-3, procaspase-8, procaspase-9, cytochrome-c, Smac/Diablo, AIF, or endonuclease G.

13. A method of treating an autoimmune disease, comprising contacting a cell of lymphoid origin with a compound that modulates the expression or activity of an Ian gene or protein, such that apoptosis is modulated and the autoimmune disease is treated.

14. The method of claim 13, wherein the autoimmune disease is diabetes.

15. The method of claim 13, wherein the Ian gene or protein is Ian4.

16. The method of claim 15, wherein the expression or activity of Ian4 is upmodulated.

17. The method of claim 13, wherein the compound modulates mitochondrial membrane permeabilization.

18. The method of claim 17, wherein the compound modulates inner mitochondrial membrane permeabilization.

19. The method of claim 13, wherein the compound modulates the release of an apoptogenic factor from mitochondria within the cell.

20. The method of claim 19, wherein the apoptogenic factor is procaspase-2, procaspase-3, procaspase-8, procaspase-9, cytochrome-c, Smac/Diablo, AIF, or endonuclease G.

21. A method of treating neoplasia, comprising contacting a neoplastic cell of lymphoid origin with a compound that modulates the expression or activity of an Ian-related gene or protein, such that apoptosis is modulated and neoplasia is treated.

22. The method of claim 21, wherein the Ian gene or protein is Ian4.

23. The method of claim 22, wherein the compound modulates mitochondrial membrane permeabilization.

24. The method of claim 23, wherein the compound modulates inner mitochondrial membrane permeabilization.

25. The method of claim 22, wherein the compound modulates the release of an apoptogenic factor from mitochondria within the cell.

26. The method of claim 25, wherein the apoptogenic factor is procaspase-2, procaspase-3, procaspase-8, procaspase-9, cytochrome-c, Smac/Diablo, AIF, or endonuclease G.

27. A method of modulating receptor-independent apoptosis, comprising contacting a cell of lymphoid origin with a compound that modulates the expression or activity of caspase 8, such that receptor-independent apoptosis is modulated.

28. The method of claim 27, wherein the cell is a T cell.

29. A method of treating an autoimmune disease, comprising contacting a cell of lymphoid origin with a compound that modulates the expression or activity of caspase 8, such that receptor-independent apoptosis is modulated and the autoimmune disease is treated.

30. The method of claim 29, wherein the autoimmune disease is diabetes.

31. A method of modulating apoptosis, comprising contacting a cell of lymphoid origin with a compound that modulates the expression or activity of the leucine-rich protein of 130 kD, hsp60, GRP78 or GRP94, such that apoptosis is modulated.

32. A method of treating an autoimmune disease, comprising contacting a cell of lymphoid origin with a compound that modulates the expression or activity of leucine rich protein of 130 kD, hsp60, GRP78 or GRP94, such that apoptosis is modulated and the autoimmune disease is treated.

33. A method of treating neoplasia, comprising contacting a cell of lymphoid origin with a compound that modulates the expression or activity of leucine rich protein of 130 kD, hsp60, GRP78 or GRP94, such that apoptosis is modulated and neoplasia is treated.

34. A method for identifying a compound that modulates the expression or anti-apoptotic activity of an Ian gene or protein, comprising:
contacting a cell expressing a functional or nonfuctional Ian gene or protein with a test compound and;
determining the ability of the test compound to modulate the expression or anti-apoptotic activity of the Ian gene or protein to thereby identify a compound that modulates the expression or anti-apoptotic activity of the Ian gene or protein.

35. The method of claim 34, wherein the Ian gene or protein is Ian4.

36. The method of claim 34, further comprising subjecting the cell to a signal that induces apoptosis.

37. The method of claim 34, wherein the ability of the test compound to modulate the expression or anti-apoptotic activity of the Ian gene or protein 4 is determined by measuring apoptosis in the cell.

38. The method of claim 34, wherein the ability of the test compound to modulate the expression or anti-apoptotic activity of the Ian gene or protein is determined by measuring a change in the membrane permeabilization of mitochondrial membranes in the cell.

39. The method of claim 38, wherein the permeabilization of the inner mitochondrial membrane is measured.

40. The method of claim 39, wherein the membrane permeabilization of mitochondrial inner membranes is measured by determining mitochondrial inner membrane potential.

41. The method of claim 34, wherein the ability of the test compound to modulate the expression or anti-apoptotic activity of the Ian gene or protein is determined by measuring the release of an apoptogenic factor from mitochondria within the cell.

42. The method of claim 41, wherein the apoptogenic factor is procaspase-2, procaspase-3, procaspase-8, procaspase-9, cytochrome-c, Smac/Diablo, AIF, or endonuclease G.

43. The method of claim 34, wherein the cell is engineered to express a heterologous nucleic acid molecule encoding a functional or non-functional Ian protein.

44. A method of identifying compounds which modulate apoptosis in a cell of lymphoid origin, comprising:
contacting a cell which expresses a functional or non-functional form of an Ian-protein with a compound;
determining the effect of the compound on apoptosis, to thereby identify a compound that modulates apoptosis in the cell.

45. The method of claim 44 wherein the Ian protein is Ian4.

46. The method of claim 44, wherein apoptosis is increased.

47. The method of claim 44, wherein apoptosis is decreased.

48. The method of claim 44, further comprising subjecting the cell to a signal that induces apoptosis.

49. The method of claim 44, wherein the ability of the test compound to modulate apoptosis is determined by measuring apoptosis in the cell.

50. The method of claim 44, wherein the ability of the test compound to modulate apoptosis is determined by measuring a change in the membrane permeabilization of mitochondria in the cell.

51. The method of claim 44, wherein the permeabilization of the inner mitochondrial membrane is measured.

52. The method of claim 44, wherein the membrane permeabilization of mitochondrial membranes is measured by determining mitochondrial inner membrane potential.

53. The method of claim 34, wherein the ability of the test compound to modulate apoptosis is determined by measuring the release of an apoptogenic factor from mitochondria within the cell.

54. The method of claim 53, wherein the apoptogenic factor is procaspase-2, procaspase-3, procaspase-8, procaspase-9, cytochrome-c, Smac/Diablo, AIF, or endonuclease G.

55. The method of claim 34, wherein the ability of the test compound to modulate apoptosis is determined by measuring the expression or activity of a component of the receptor-independent apoptotic pathway.

56. The method of claim 34, wherein the ability of the test compound to modulate apoptosis is determined by measuring the expression or activity of the leucine-rich protein of 130 kD, hsp60, GRP78 or GRP94.

57. A method for identifying a compound that modulates the receptor-independent pro-apoptotic activity of caspase 8, comprising:
contacting a cell expressing caspase 8 with a test compound and;
determining the ability of the test compound to modulate the receptor-independent pro-apoptotic activity of caspase 8 to thereby identify a compound that modulates the receptor-independent pro-apoptotic activity of caspase 8.

58. The method of claim 57, wherein the ability of the test compound to modulate the receptor-independent pro-apoptotic activity of caspase 8 is determined by measuring apoptosis in the cell.

59. The method of claim 58, wherein the ability of the test compound to modulate the receptor-independent pro-apoptotic activity of caspase 8 is determined by measuring a change in the membrane permeabilization of mitochondrial membranes in the cell.

60. The method of claim 59, wherein the permeabilization of the inner mitochondrial membrane is measured.

61. The method of claim 60, wherein the membrane permeabilization of mitochondrial inner membranes is measured by determining mitochondrial inner membrane potential.

62. The method of claim 57, wherein the ability of the test compound to modulate apoptosis is determined by measuring the release of an apoptogenic factor from mitochondria within the cell.

63. The method of claim 62, wherein the apoptogenic factor is procaspase-2, procaspase-3, procaspase-8, procaspase-9, cytochrome-c, Smac/Diablo, AIF, or endonuclease G.

64. The method of claim 57, wherein the ability of the test compound to modulate apoptosis is determined by measuring the expression or activity of a component of the recertor-independent apoptotic pathway.

65. The method of claim 64, wherein the ability of the test compound to modulate apoptosis is determined by measuring the expression or activity of the leucine rich protein of 130 kD, hsp60, GRP78 or GRP94.

66. A method for identifying a compound that modulates the expression or anti-apoptotic activity of an Ian gene or protein, comprising:
administering a test compound to a model organism with a mutant Ian phenotype;
determining the ability of the test compound to rescue the mutant Ian phenotype to thereby identify a compound that modulates the expression or anti-apoptotic activity of the Ian gene or protein.

67. The method of claim 66, wherein the Ian gene or protein is Ian4.

68. The method of claim 66, further comprising subjecting the organism to a signal that induces apoptosis.

69. A method of identifying a compound that modulates apoptosis in a cell of lymphoid origin, comprising:
administering a test compound to an animal model with a mutant Ian phenotype; determining the ability of the test compound to rescue the mutant Ian phenotype, to thereby identify a compound that modulates apoptosis in the cell.

70. The method of claim 69, wherein the Ian protein is Ian4.

71. The method of claim 69, further comprising subjecting the animal model to a signal that induces apoptosis.

72. A method for identifying a compound capable of treating an autoimmune disorder characterized by lymphopenia, comprising:
administering a test compound to a animal model with a mutant Ian phenotype; determining the ability of the test compound to rescue the mutant Ian phenotype, to thereby identify a compound capable of treating a disorder characterized by lymphopenia.

73. The method of claim 72, wherein the Ian protein is Ian4.

74. The method of claim 72, further comprising subjecting the animal model to a signal that induces apoptosis.

75. The method of claim 72, wherein the disorder is diabetes.

76. A method for identifying a compound capable of treating a neoplastic disorder of lymphoid origin characterized by aberrant Ian expression, comprising:
administering a test compound to a non-human transgenic organism capable of overexpressing an Ian protein; determining the ability of the test compound to rescue the mutant Ian phenotype, to thereby identify a compound capable of treating a neoplastic disorder of lymphoid origin.

77. The method of claim 76, wherein the Ian protein is Ian4.

78. The method of claim 72, further comprising subjecting the organism to a signal that induces apoptosis.

79. The method of claim 72, wherein the neoplastic disorder is leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burkett's lymphoma.

80. A method for determining if a subject is at risk for an autoimmune disorder characterized by lymphopenia comprising detecting, in a sample of cells from the subject, the presence or absence of a genetic lesion, wherein the genetic lesion is characterized by an alteration affecting the misexpression of the Ian gene.

81. The method of claim 80, wherein the Ian protein is Ian4.

82. The method of claim 81, wherein the disorder is diabetes.

83. A method for determining if a subject is at risk for a neoplastic disorder characterized by Ian overexpression comprising detecting, in a sample of cells from the subject, the presence or absence of a genetic lesion, wherein the genetic lesion is characterized by an alteration affecting the integrity of a gene encoding an Ian protein or overexpression of the Ian gene.

84. The method of claim 83, wherein the Ian protein is Ian4.

85. The method of claim 81, wherein the disorder is leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burkett's lymphoma.
